(19) United States
(12) Patent Application Publication
(10) Pub. No.: US 2025/0340879 A1
(43) Pub. Date: Nov. 6, 2025

(54) COMPOSITIONS AND METHODS FOR NUCLEIC ACID TRANSFER

(71) Applicant: Children's Hospital Medical Center, Cincinnati, OH (US)

(72) Inventors: Assem G. Ziady, Newport, KY (US); Matthew Siefert, Cincinnati, OH (US); Songbai Lin, Lawrenceville, GA (US); Harrison Brown, Atlanta, GA (US)

(21) Appl. No.: 19/266,565

(22) Filed: Jul. 11, 2025

Related U.S. Application Data
(62) Division of application No. 17/121,835, filed on Dec. 15, 2020, now Pat. No. 12,378,550.
Application No. 17/121,835 is a continuation of application No. 15/690,526, filed on Aug. 30, 2017, now Pat. No. 10,894,960.
(60) Provisional application No. 62/381,237, filed on Aug. 30, 2016.

(51) Int. Cl. C12N 15/113; A61K 31/05; A61K 31/132; A61K 31/265; A61K 31/352; A61K 31/395; A61K 31/4709; A61K 31/519; A61K 31/52; A61K 31/565; A61K 31/573; A61K 31/704; A61K 31/7048; A61K 31/7088; A61K 31/713; A61K 35/28; A61K 38/17; A61K 45/06; A61K 47/60; A61K 47/64; A61K 48/00; C12N 15/11; C12N 15/85

(52) U.S. Cl. CPC C12N 15/113; A61K 31/05; A61K 31/132; A61K 31/265; A61K 31/352; A61K 31/395; A61K 31/4709; A61K 31/519; A61K 31/52; A61K 31/565; A61K 31/573; A61K 31/704; A61K 31/7048; A61K 31/7088; A61K 31/713; A61K 38/1709; A61K 45/06; A61K 47/60; A61K 47/6455; A61K 48/0008; A61K 48/0066; C12N 15/111; C12N 15/85; A61K 35/28; C12N 2310/14; C12N 2320/31; C12N 2320/32

(57) ABSTRACT

Disclosed are methods for the enhancement of nucleic acid delivery systems. The methods may employ treatment with a compound and/or an RNAi molecule in combination with a nucleic acid to improve nucleic acid uptake into a cell. In particular, the disclosed methods may be useful for improved gene therapy techniques.

### CROSS REFERENCE TO RELATED APPLICATION

This application claims priority to and benefit of U.S. Provisional Patent Application Ser. No. 62/381,237, filed Aug. 30, 2016, entitled “Method for Enhancing Nucleic Acid Transfer,” the contents of which are incorporated by reference in its entirety for all purposes.

### BACKGROUND

Transfer of nucleic acids, including double and single stranded DNA as well as RNA, into eukaryotic cells is the most essential step of any gene transfer, repair, or editing technology. Transfer of nucleic acids may be accomplished using many types of delivery vehicles, including cationic lipids, viral vectors and nucleic acid nanoparticles condensed with cationic polymers such as poly lysine or polyethyleneimine. However, significant costs involved in the preparation of these materials present a significant limitation in their usage as both research tools and translational applications such as gene therapy. Further, efficacy of nucleic acid transfer with or without modification of the vector remains an area in need of improvement. The instant disclosure seeks to address one or more of the aforementioned needs in the art.

### BRIEF SUMMARY

Disclosed are methods for the enhancement of nucleic acid delivery systems. The methods may employ treatment with a compound and/or an RNAi molecule in combination with, for example, prior to or concurrent with, administration of a nucleic acid to improve nucleic acid uptake into a cell. In particular, the disclosed methods may be useful for improved gene therapy techniques in which a disclosed RNAi and/or a disclosed compound may be administered prior to or concurrently with the gene therapy delivery vehicle containing a nucleic acid.

### BRIEF DESCRIPTION OF THE DRAWINGS

FIG. 1 is a schematic of partial NNP (DNP and RNP) interactome including nucleoin, APC, and SPTAN1, which were identified by MS analysis of 2 gel bands from DNP and RNP pull downs not present in bead alone control. Lighter color circles connote interactions that enhance NNP-mediated gene transfer, while darker circles connote interactions that inhibit. (+) or (−) along arrows connote impact on interactions with DNP. (+) or (−) by pharmacological agents reflect impact on the activation of GR, CDK1, or CKII. For example, while cortisone would increase nucleolin at the membrane via GR (10), spermine would increase it through stimulation of CKII mediated phosphorylation of nucleolin. Pull downs initially conducted in primary hepatocytes and repeated three times in wd-AECs for 2 non-CF and 3 CF subjects. This DNP interactome was observed in all the hepatocyte and CF and non-CF wd-AEC studies.

FIG. 2 depicts immunoprecipitation of protein interactors of DNA nanoparticles in HeLa cells.

FIG. 3 depicts enhanced DNA nanoparticle transfection through siRNA expression.

FIG. 4 depicts transfection of human primary airway epithelia either following prior treatment with scrambled shRNA, shRNA specific for APC, or shRNA specific for SPTAN1 for 48 hours. Luciferase expression was measured two days post transfection. * connotes different from saline pretreatment (triplicates in three experiments p<0.01).

FIG. 5 depicts primary cell cultures of airway epithelia transfected with DNPs containing a plasmid coding for luciferase driven by the ubiquitin B promoter (5.4 kb). shRNA lentivirus infection was 48 hours prior to transfection while spermine (CK11 inducer) roscovitine (CDK1 inhibitor), resveratrol (CDK1 agonist), or cortisone (GR agonist) were added four hours prior to transfection. Treatments were saline(S), APC shRNA (-APC), SPTAN1 shRNA (-SPTAN1), hydrocortisone©, spermine (Sper), roscovitine (Ros), or resveratrol (RES). Luciferase expression was measured two days post transfection. * connotes different from saline (p<0.01).

### DETAILED DESCRIPTION OF THE INVENTION

### Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms and expressions used herein have the ordinary meaning as is accorded to such terms and expressions with respect to their corresponding respective areas of inquiry and study except where specific meanings have otherwise been set forth herein.

As used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of up to 20%, or up to 10%, or up to 5%, or up to 1% of a given value. Alternatively, particularly with respect to biological systems or processes, the term can mean within an order of magnitude, for example, within 5-fold, and or for example, within 2-fold, of a value. Where particular values are described in the application and claims, unless otherwise stated the term “about” meaning within an acceptable error range for the particular value should be assumed.

The terms “individual,” “host,” “subject,” and “patient” are used interchangeably to refer to an animal that is the object of treatment, observation and/or experiment. Generally, the term refers to a human patient, but the methods and compositions may be equally applicable to non-human subjects such as other mammals. In some embodiments, the terms refer to humans. In further embodiments, the terms may refer to children.

The terms “treat,” “treating” or “treatment,” as used herein, refers to methods of alleviating, abating or ameliorating a disease or condition symptoms, preventing additional symptoms, ameliorating or preventing the underlying metabolic causes of symptoms, inhibiting the disease or condition, arresting the development of the disease or condition, relieving the disease or condition, causing regression of the disease or condition, relieving a condition caused by the disease or condition, or stopping the symptoms of the disease or condition either prophylactically and/or therapeutically.

The term “pharmaceutically acceptable,” as used herein, refers a material, such as a carrier or diluent, which does not abrogate the biological activity or properties of the compounds described herein. Such materials are administered to an individual without causing undesirable biological effects or interacting in a deleterious manner with any of the components of the composition in which it is contained.

The term “pharmaceutically acceptable salt,” as used herein, refers to a formulation of a compound that does not cause significant irritation to an organism to which it is administered and does not abrogate the biological activity and properties of the compounds described herein. As used herein, the disclosed compounds also include pharmaceutically acceptable salts thereof.

The terms “composition” or “pharmaceutical composition,” as used herein, refers to a mixture of at least one compound or RNAi as disclosed herein, with at least one and optionally more than one other pharmaceutically acceptable chemical components, such as carriers, stabilizers, diluents, dispersing agents, suspending agents, thickening agents, and/or excipients.

The term “carrier” applied to pharmaceutical compositions of the disclosure refers to a diluent, excipient, or vehicle with which an active compound (e.g., dextromethorphan) is administered. Such pharmaceutical carriers can be sterile liquids, such as water, saline solutions, aqueous dextrose solutions, aqueous glycerol solutions, and oils, including those of petroleum, animal, vegetable, or synthetic origin, such as peanut oil, soybean oil, mineral oil, sesame oil and the like.

The term “NNP” refers to a Nucleic acid Nano Particle: a complex of DNA or RNA with polymers of lysines (15-45 lysines long)

The term “DNP” refers to a DNA Nanoparticle

The term “RNP” refers to a RNA Nanoparticle

The term “Interactome” refers to the whole set of molecular interactions in a particular cell. The term specifically refers to physical interactions among molecules (such as those among proteins, also known as protein-protein interactions) but can also describe sets of indirect interactions among genes (genetic interactions).

The term “APC” refers to an adenomatous polyposis coli protein

The term “wd-AECs” refer to well-differentiated airway epithelial cells.

The term “SPTAN1” refers to Alpha II-spectrin, also known as Spectrin alpha chain, a protein that in humans is encoded by the SPTAN1 gene. Alpha II-spectrin is expressed in a variety of tissues, and is highly expressed in cardiac muscle at Z-disc structures, costameres and at the sarcolemma membrane.

The term “GR” refers to a glucocorticoid receptor

The term “CDK1” refers to cyclin dependent kinase 1

The term “CKII” refers to casein kinase II

The term “Spermine” refers to a polyamine involved in cellular metabolism found in all eukaryotic cells.

The term “shRNA” refers to a small hairpin RNA or short hairpin RNA (shRNA) is an artificial RNA molecule with a tight hairpin turn that can be used to silence target gene expression via RNA interference (RNAi)

Disclosed herein are methods for the enhancement of nucleic acid delivery systems by combination treatment with one or more compounds as disclosed herein and/or one or more RNAi molecules as disclosed herein. For example, the disclosed methods may be used with delivery of a nucleic acid such as a gene, a gene fragment, a fragment containing an active portion of a protein encoded by a gene, or the like. Further examples of nucleic acids that may be delivered include nucleic acid components of the CRISPR/CAS9, or short nucleic acids, such as microRNA or DNA or RNA oligonucleotides. The disclosed RNAi molecules and/or compounds may be administered to an individual in need of administration of a nucleic acid prior to administration of a nucleic acid delivery system, or concurrently with the administration of a nucleic acid delivery system.

The method, in certain aspects, may comprise the steps of
contacting a cell with an RNAi molecule or an active agent. The RNAi molecule or active agent may be in an amount sufficient to inhibit synthesis of one or more proteins that inhibit nucleic acid delivery vehicle uptake; and
contacting the eukaryotic cell with a nucleic acid delivery vehicle.

The cell may be, for example, a eukaryotic cell, derived from a human being.

In one aspect, a method of treating an individual is disclosed. The individual may be one in which administration a therapeutically effective amount of a protein may be advantageous to reversal, prevention, or amelioration of a disease state. The delivery of a protein may be achieved via administration of a gene, or portion of a gene that encodes an active portion of a protein, that may be subsequently expressed in the individual to provide a functional protein or functional protein fragment in a therapeutically effective amount. In this aspect, the method may comprise the steps of administering an RNAi that inhibits expression of a gene encoding a protein selected from a protein of Table 1 and/or a compound selected from Table 2 or 3, concurrently, before, or after administration of a drug delivery vehicle containing the nucleic acid that encodes the gene, or in some instances, the active portion of a gene, of interest.

The amount of compound and/or RNAi necessary to effect the methods of the instant disclosure may be determined by one of ordinary skill in the art. The dose administered to a subject, particularly a human, may be sufficient to effect the desired response in the subject over a reasonable period of time. The dose may be determined by the strength of the particular compound employed and the condition of the subject, as well as the body weight of the subject to be treated. The existence, nature, and extent of any adverse side effects that might accompany the administration of a particular compound also will determine the size of the dose and the particular route of administration employed with a particular patient. For example, the compounds may be therapeutically effective at low doses. Exemplary dosage ranges may be from about 0.001 mM, or less, to about 100 mM, or more, or from about 0.01, 0.05, 0.1, 0.5, 0.6, 0.7, 0.8, or 0.9 mM, to about 1, 2, 3, 4, 5, 6, 7, 8, 9, 10, 20, 30, 40 50, 60, 70, 80, 90 or 100 mM. Accordingly, the compounds may be generally administered in low doses.

In one aspect, the gene is the CF gene, and the individual in need of treatment is an individual having cystic fibrosis.

In one aspect, the RNAi molecule may be one that inhibits expression of a gene encoding a protein selected from a protein of Table 1.

Table 1. Genes encoding proteins that modulate nucleic acid delivery vehicle uptake. The RNAi molecules of the instant disclosure may inhibit expression of one or more of the genes listed in the table.

| | Gene | |
|---|---|---|
| Uniprot ID | names | Protein names |
| P04114 | APOB | Apolipoprotein B-100 (Apo B-100) [Cleaved into: |
| | | Apolipoprotein B-48 (Apo B-48)] |
| P29536 | LMOD1 | Leiomodin-1 (64 kDa autoantigen 1D) (64 kDa |
| | | autoantigen 1D3) (64 kDa autoantigen D1) (Leiomodin, |
| | | muscle form) (Smooth muscle leiomodin) (SM-Lmod) |
| | | (Thyroid-associated ophthalmopathy autoantigen) |
| P68104 | EEF1A1 | Elongation factor 1-alpha 1 (EF-1-alpha-1) (Elongation |
| | EEF1A | factor Tu) (EF-Tu) (Eukaryotic elongation factor 1 A-1) |
| | EF1A | (eEF1A-1) (Leukocyte receptor cluster member 7) |
| | LENG7 | |
| O19680 | | Pot. HLA-DP-alpha 1 (Aa −31 to +2) (441 is 1st base in |
| | | codon) (Fragment) |
| P46939 | UTRN | Utrophin (Dystrophin-related protein 1) (DRP-1) |
| | DMDL | |
| | DRP1 | |
| P08590 | MYL3 | Myosin light chain 3 (Cardiac myosin light chain 1) |
| | | (CMLC1) (Myosin light chain 1, slow-twitch muscle |
| | | B/ventricular isoform) (MLC1SB) (Ventricular myosin |
| | | alkali light chain) (Ventricular myosin light chain 1) |
| | | (VLCl) (Ventricular/slow twitch myosin alkali light |
| | | chain) (MLC-lV/sb) |
| P22695 | UQCRC2 | Cytochrome b-c1 complex subunit 2, mitochondrial |
| | | (Complex III subunit 2) (Core protein II) (Ubiquinol- |
| | | cytochrome-c reductase complex core protein 2) |
| Q16763 | UBE2S | Ubiquitin-conjugating enzyme E2 S (EC 2.3.2.23) (E2 |
| | E2EPF | ubiquitin-conjugating enzyme S) (E2-EPF) (Ubiquitin |
| | OK/SW- | carrier protein S) (Ubiquitin-conjugating enzyme E2-24 |
| | cl.73 | kDa) (Ubiquitin-conjugating enzyme E2-EPF5) |
| | | (Ubiquitin-protein ligase S) |
| P00451 | F8 F8C | Coagulation factor VIII (Antihemophilic factor) (AHF) |
| | | (Procoagulant component) [Cleaved into: Factor VIIIa |
| | | heavy chain, 200 kDa isoform; Factor VIIIa heavy chain, |
| | | 92 kDa isoform; Factor VIII B chain; Factor VIIIa light |
| | | chain] |
| P52272 | HNRNPM | Heterogeneous nuclear ribonucleoprotein M (hnRNP M) |
| | HNRPM | |
| | NAGR1 | |
| P60660 | MYL6 | Myosin light polypeptide 6 (17 kDa myosin light chain) |
| | | (LC17) (Myosin light chain 3) (MLC-3) (Myosin light |
| | | chain alkali 3) (Myosin light chain A3) (Smooth muscle |
| | | and nonmuscle myosin light chain alkali 6) |
| P25054 | APC DP2.5 | Adenomatous polyposis coli protein (Protein APC) |
| | | (Deleted in polyposis 2.5) |
| P23458 | JAK1 | Tyrosine-protein kinase JAK1 (EC 2.7.10.2) (Janus |
| | JAK1A | kinase 1) (JAK-1) |
| | JAK1B | |
| P13533 | MYH6 | Myosin-6 (Myosin heavy chain 6) (Myosin heavy chain, |
| | MYHCA | cardiac muscle alpha isoform) (MyHC-alpha) |
| P61247 | RPS3A | 40S ribosomal protein S3a (Small ribosomal subunit |
| | FTE1 | protein eS1) (v-fos transformation effector protein) (Fte- |
| | MFTL | 1) |
| Q08379 | GOLGA2 | Golgin subfamily A member 2 (130 kDa cis-Golgi |
| | | matrix protein) (GM130) (GM130 autoantigen) (Golgin- |
| | | 95) |
| P41219 | PRPH | Peripherin (Neurofilament 4) |
| | NEF4 | |
| | PRPH1 | |
| Q99729 | HNRNPAB | Heterogeneous nuclear ribonucleoprotein A/B (hnRNP |
| | ABBP1 | A/B) (APOBEC1-binding protein 1) (ABBP-1) |
| | HNRPAB | |
| P11277 | SPTB | Spectrin beta chain, erythrocytic (Beta-I spectrin) |
| | SPTB1 | |
| P33981 | TTK MPS1 | Dual specificity protein kinase TTK (EC 2.7.12.1) |
| | MPS1L1 | (Phosphotyrosine picked threonine-protein kinase) (PYT) |
| P11021 | HSPA5 | 78 kDa glucose-regulated protein (GRP-78) |
| | GRP78 | (Endoplasmic reticulum lumenal Ca(2+)-binding protein |
| | | grp78) (Heat shock 70 kDa protein 5) (Immunoglobulin |
| | | heavy chain-binding protein) (BiP) |
| Q15552 | tb protein | CACCC box-binding protein |
| P62913 | RPL11 | 60S ribosomal protein L11 (CLL-associated antigen |
| | | KW-12) (Large ribosomal subunit protein uL5) |
| P38919 | EIF4A3 | Eukaryotic initiation factor 4A-III (eIF-4A-III) (eIF4A- |
| | DDX48 | III) (EC 3.6.4.13) (ATP-dependent RNA helicase |
| | KIAA0111 | DDX48) (ATP-dependent RNA helicase eIF4A-3) |
| | | (DEAD box protein 48) (Eukaryotic initiation factor 4A- |
| | | like NUK-34) (Eukaryotic translation initiation factor 4A |
| | | isoform 3) (Nuclear matrix protein 265) (NMP 265) |
| | | (hNMP 265) [Cleaved into: Eukaryotic initiation factor |
| | | 4A-III, N-terminally processed] |
| Q12905 | ILF2 NF45 | Interleukin enhancer-binding factor 2 (Nuclear factor of |
| | PRO3063 | activated T-cells 45 kDa) |
| Q14978 | NOLC1 | Nucleolar and coiled-body phosphoprotein 1 (140 kDa |
| | KIAA0035 | nucleolar phosphoprotein) (Nopp140) (Hepatitis C virus |
| | NS5ATP13 | NS5A-transactivated protein 13) (HCV NS5A- |
| | | transactivated protein 13) (Nucleolar 130 kDa protein) |
| | | (Nucleolar phosphoprotein p130) |
| P20929 | NEB | Nebulin |
| Q16296 | 4R-MAP2 | Microtubule-associated protein (Fragment) |
| P33991 | MCM4 | DNA replication licensing factor MCM4 (EC 3.6.4.12) |
| | CDC21 | (CDC21 homolog) (P1-CDC21) |
| P49454 | CENPF | Centromere protein F (CENP-F) (AH antigen) |
| | | (Kinetochore protein CENPF) (Mitosin) |
| Q14008 | CKAP5 | Cytoskeleton-associated protein 5 (Colonic and hepatic |
| | KIAA0097 | tumor overexpressed gene protein) (Ch-TOG) |
| Q14839 | CHD4 | Chromodomain-helicase-DNA-binding protein 4 (CHD- |
| | | 4) (EC 3.6.4.12) (ATP-dependent helicase CHD4) (Mi-2 |
| | | autoantigen 218 kDa protein) (Mi2-beta) |
| P55017 | SLC12A3 | Solute carrier family 12 member 3 (Na—Cl cotransporter) |
| | NCC TSC | (NCC) (Na—Cl symporter) (Thiazide-sensitive sodium- |
| | | chloride cotransporter) |
| Q92835 | INPP5D | Phosphatidylinositol 3,4,5-trisphosphate 5-phosphatase 1 |
| | SHIP SHIP1 | (EC 3.1.3.86) (Inositol polyphosphate-5-phosphatase of |
| | | 145 kDa) (SIP-145) (SH2 domain-containing inositol 5′- |
| | | phosphatase 1) (SH2 domain-containing inositol |
| | | phosphatase 1) (SHIP-1) (p150Ship) (hp51CN) |
| Q15269 | PWP2 | Periodic tryptophan protein 2 homolog |
| | PWP2H | |
| P20585 | MSH3 | DNA mismatch repair protein Msh3 (hMSH3) |
| | DUC1 DUG | (Divergent upstream protein) (DUP) (Mismatch repair |
| | | protein 1) (MRP1) |
| Q05086 | UBE3A | Ubiquitin-protein ligase E3A (EC 2.3.2.26) (E6AP |
| | E6AP | ubiquitin-protein ligase) (HECT-type ubiquitin |
| | EPVE6AP | transferase E3A) (Human papillomavirus E6-associated |
| | HPVE6A | protein) (Oncogenic protein-associated protein E6-AP) |
| | | (Renal carcinoma antigen NY-REN-54) |
| Q92922 | SMARCC1 | SWI/SNF complex subunit SMARCC1 (BRG1- |
| | BAF155 | associated factor 155) (BAF155) (SWI/SNF complex |
| | | 155 kDa subunit) (SWI/SNF-related matrix-associated |
| | | actin-dependent regulator of chromatin subfamily C |
| | | member 1) |
| P62807 | HIST1H2B | Histone H2B type 1-C/E/F/G/I (Histone H2B.1 A) |
| | C H2BFL; | (Histone H2B.a) (H2B/a) (Histone H2B.g) (H2B/g) |
| | HIST1H2B | (Histone H2B.h) (H2B/h) (Histone H2B.k) (H2B/k) |
| | E H2BFH; | (Histone H2B.l) (H2B/l) |
| | HIST1H2B | |
| | F H2BFG; | |
| | HIST1H2B | |
| | G H2BFA; | |
| | HIST1H2BI | |
| | H2BFK | |
| Q92800 | EZH1 | Histone-lysine N-methyltransferase EZH1 (EC 2.1.1.43) |
| | KIAA0388 | (ENX-2) (Enhancer of zeste homolog 1) |
| P78549 | NTHL1 | Endonuclease III-like protein 1 (hNTH1) (EC 3.2.2.—) |
| | NTH1 | (EC 4.2.99.18) (Bifunctional DNA N-glycosylase/DNA- |
| | OCTS3 | (apurinic or apyrimidinic site) lyase) (DNA |
| | | glycosylase/AP lyase) |
| Q12789 | GTF3C1 | General transcription factor 3C polypeptide 1 (TF3C- |
| | | alpha) (TFIIIC box B-binding subunit) (Transcription |
| | | factor IIIC 220 kDa subunit) (TFIIIC 220 kDa subunit) |
| | | (TFIIIC220) (Transcription factor IIIC subunit alpha) |
| O14686 | KMT2D | Histone-lysine N-methyltransferase 2D (Lysine N- |
| | ALR MLL2 | methyltransferase 2D) (EC 2.1.1.43) (ALL1-related |
| | MLL4 | protein) (Myeloid/lymphoid or mixed-lineage leukemia |
| | | protein 2) |
| Q13304 | GPR17 | Uracil nucleotide/cysteinyl leukotriene receptor |
| | | (UDP/CysLT receptor) (G-protein coupled receptor 17) |
| | | (P2Y-like receptor) (R12) |
| Q9UQB3 | CTNND2 | Catenin delta-2 (Delta-catenin) (GT24) (Neural |
| | NPRAP | plakophilin-related ARM-repeat protein) (NPRAP) |
| | | (Neurojungin) |
| P30519 | HMOX2 | Heme oxygenase 2 (HO-2) (EC 1.14.14.18) |
| | HO2 | |
| O60437 | PPL | Periplakin (190 kDa paraneoplastic pemphigus antigen) |
| | KIAA0568 | (195 kDa cornified envelope precursor protein) |
| Q15413 | RYR3 | Ryanodine receptor 3 (RYR-3) (RyR3) (Brain ryanodine |
| | HBRR | receptor-calcium release channel) (Brain-type ryanodine |
| | | receptor) (Type 3 ryanodine receptor) |
| Q13618 | CUL3 | Cullin-3 (CUL-3) |
| | KIAA0617 | |
| O75691 | UTP20 | Small subunit processome component 20 homolog |
| | DRIM | (Down-regulated in metastasis protein) (Novel nucleolar |
| | | protein 73) (NNP73) (Protein Key-1A6) |
| O80743 | T13D8.9 | T13D8.9 protein |
| P38159 | RBMX | RNA-binding motif protein, X chromosome |
| | HNRPG | (Glycoprotein p43) (Heterogeneous nuclear |
| | RBMXP1 | ribonucleoprotein G) (hnRNP G) [Cleaved into: RNA- |
| | | binding motif protein, X chromosome, N-terminally |
| | | processed] |
| O75081 | CBFA2T3 | Protein CBFA2T3 (MTG8-related protein 2) (Myeloid |
| | MTG16 | translocation gene on chromosome 16 protein) |
| | MTGR2 | (hMTG16) (Zinc finger MYND domain-containing |
| | ZMYND4 | protein 4) |
| O95153 | TSPOAP1 | Peripheral-type benzodiazepine receptor-associated |
| | BZRAP1 | protein 1 (PRAX-1) (Peripheral benzodiazepine receptor- |
| | KIAA0612 | interacting protein) (PBR-IP) (RIMS-binding protein 1) |
| | RBP1 | (RIM-BP1) (TSPO-associated protein 1) |
| | RIMBP1 | |
| P63267 | ACTG2 | Actin, gamma-enteric smooth muscle (Alpha-actin-3) |
| | ACTA3 | (Gamma-2-actin) (Smooth muscle gamma-actin) |
| | ACTL3 | |
| | ACTSG | |
| P18754 | RCC1 | Regulator of chromosome condensation (Cell cycle |
| | CHC1 | regulatory protein) (Chromosome condensation protein |
| | | 1) |
| Q5T081 | RCC1 | CHC1 protein (Regulator of chromosome condensation 1 |
| | CHC1 | isoform 1) (Regulator of chromosome condensation 1, |
| | hCG_27809 | isoform CRA_b) |
| P13639 | EEF2 EF2 | Elongation factor 2 (EF-2) |
| Q16695 | HIST3H3 | Histone H3.1t (H3/t) (H3t) (H3/g) |
| | H3FT | |
| A8K401 | PHB | Prohibitin, isoform CRA_a (cDNA FLJ78511, highly |
| | hCG_29613 | similar to Homo sapiens prohibitin (PHB), mRNA) |
| | | (cDNA, FLJ93035, Homo sapiens prohibitin (PHB), |
| | | mRNA) |
| P35232 | PHB | Prohibitin |
| Q53FV0 | | Prohibitin variant (Fragment) |
| P83731 | RPL24 | 60S ribosomal protein L24 (60S ribosomal protein L30) |
| | | (Large ribosomal subunit protein eL24) |
| V9HW01 | HEL-S-310 | Epididymis secretory protein Li 310 |
| A0A024RCA7 | RPLP2 | Ribosomal protein, large, P2, isoform CRA_a |
| | hCG_1778304 | |
| P05387 | RPLP2 | 60S acidic ribosomal protein P2 (Large ribosomal |
| | D11S2243E | subunit protein P2) (Renal carcinoma antigen NY-REN- |
| | RPP2 | 44) |
| P46783 | RPS10 | 40S ribosomal protein S10 (Small ribosomal subunit |
| | | protein eS10) |
| P62280 | RPS11 | 40S ribosomal protein S11 (Small ribosomal subunit |
| | | protein uS17) |
| P62277 | RPS13 | 40S ribosomal protein S13 (Small ribosomal subunit |
| | | protein uS15) |
| P08708 | RPS17 | 40S ribosomal protein S17 (Small ribosomal subunit |
| | RPS17L | protein eS17) |
| A8K517 | RPS23 | Ribosomal protein S23, isoform CRA_a (cDNA |
| | hCG_38189 | FLJ77921, highly similar to Homo sapiens ribosomal |
| | | protein S23 (RPS23), mRNA) (cDNA, FLJ92033, Homo sapiens |
| | | ribosomal protein S23 (RPS23), mRNA) |
| P62266 | RPS23 | 40S ribosomal protein S23 (Small ribosomal subunit |
| | | protein uS12) |
| P62851 | RPS25 | 40S ribosomal protein S25 (Small ribosomal subunit |
| | | protein eS25) |
| B2R491 | RPS4X | 40S ribosomal protein S4 |
| | hCG_18634 | |
| P62701 | RPS4X | 40S ribosomal protein S4, X isoform (SCR10) (Single |
| | CCG2 RPS4 | copy abundant mRNA protein) (Small ribosomal subunit |
| | SCAR | protein eS4) |
| P62241 | RPS8 | 40S ribosomal protein S8 (Small ribosomal subunit |
| | OK/SW- | protein eS8) |
| | cl.83 | |
| Q5JR94 | RPS8 | 40S ribosomal protein S8 |
| | hCG_2031852 | |
| P12755 | SKI | Ski oncogene (Proto-oncogene c-Ski) |
| A0A1L1UHR1 | | Sperm binding protein 1a |
| B3KTS5 | | cDNA FLJ38670 fis, clone HSYRA2000190, highly |
| | | similar to Voltage-dependent anion-selective channel |
| | | protein 1 |
| P21796 | VDAC1 | Voltage-dependent anion-selective channel protein 1 |
| | VDAC | (VDAC-1) (hVDAC1) (Outer mitochondrial membrane |
| | | protein porin 1) (Plasmalemmal porin) (Porin 31HL) |
| | | (Porin 31HM) |
| P25490 | YY1 | Transcriptional represser protein YY1 (Delta |
| | INO80S | transcription factor) (INO80 complex subunit S) (NF-E1) |
| | | (Yin and yang 1) (YY-1) |
| Q99996 | AKAP9 | A-kinase anchor protein 9 (AKAP-9) (A-kinase anchor |
| | AKAP350 | protein 350 kDa) (AKAP 350) (hgAKAP 350) (A-kinase |
| | AKAP450 | anchor protein 450 kDa) (AKAP 450) (AKAP 120-like |
| | KIAA0803 | protein) (Centrosome- and Golgi-localized PKN- |
| | | associated protein) (CG-NAP) (Protein hyperion) |
| | | (Protein kinase A-anchoring protein 9) (PRKA9) (Protein |
| | | yotiao) |
| P16402 | HIST1H1D | Histone H1.3 (Histone H1c) (Histone H1s-2) |
| | H1F3 | |
| Q96GY0 | ZC2HC1A | Zinc finger C2HC domain-containing protein 1A |
| | C8orf70 | |
| | FAM164A | |
| | CGI-62 | |
| P02545 | LMNA | Prelamin-A/C [Cleaved into: Lamin-A/C (70 kDa lamin) |
| | LMN1 | (Renal carcinoma antigen NY-REN-32)] |
| P20700 | LMNB1 | Lamin-B1 |
| | LMN2 | |
| | LMNB | |
| P14550 | AKR1A1 | Alcohol dehydrogenase [NADP(+)] (EC 1.1.1.2) |
| | ALDR1 | (Aldehyde reductase) (Aldo-keto reductase family 1 |
| | ALR | member A1) |
| V9HWI0 | HEL-S- | Epididymis secretory protein Li 6 (Epididymis secretory |
| | 165mP | sperm binding protein Li 165 mP) |
| | HEL-S-6 | |
| A0PJH2 | ATP5H | ATP5H protein (Fragment) |
| O75947 | ATP5H | ATP synthase subunit d, mitochondrial (ATPase subunit |
| | My032 | d) |
| P19105 | MYL12A | Myosin regulatory light chain 12A (Epididymis secretory |
| | MLCB | protein Li 24) (HEL-S-24) (MLC-2B) (Myosin RLC) |
| | MRLC3 | (Myosin regulatory light chain 2, nonsarcomeric) |
| | RLC | (Myosin regulatory light chain MRLC3) |
| A0A0G2JS52 | | Uncharacterized protein (Fragment) |
| V9H0H3 | | Gag-Pro-Pol-Env protein |
| P17096 | HMGA1 | High mobility group protein HMG-I/HMG-Y (HMG- |
| | HMGIY | I(Y)) (High mobility group AT-hook protein 1) (High |
| | | mobility group protein A1) (High mobility group protein |
| | | R) |
| O46577 | COX4I1 | Cytochrome c oxidase subunit 4 isoform 1, |
| | COX4 | mitochondrial (Cytochrome c oxidase polypeptide IV) |
| | | (Cytochrome c oxidase subunit IV isoform 1) (COX IV- |
| | | 1) (Fragment) |
| Q9UIG0 | BAZ1B | Tyrosine-protein kinase BAZ1B (EC 2.7.10.2) |
| | WBSC10 | (Bromodomain adjacent to zinc finger domain protein |
| | WBSCR10 | 1B) (Williams syndrome transcription factor) (Williams- |
| | WBSCR9 | Beuren syndrome chromosomal region 10 protein) |
| | WSTF | (Williams-Beuren syndrome chromosomal region 9 |
| | | protein) (hWALp2) |
| Q9UHD8 | SEPT9 | Septin-9 (MLL septin-like fusion protein MSF-A) (MLL |
| | KIAA0991 | septin-like fusion protein) (Ovarian/Breast septin) (Ov/Br |
| | MSF | septin) (Septin D1) |
| P62270 | Rps18 | 40S ribosomal protein S18 (Ke-3) (Ke3) |
| Q561N5 | Rps18 | MCG23000, isoform CRA_b (Putative uncharacterized |
| | mCG_23000 | protein) (Ribosomal protein S18) |
| Q9NWS8 | RMND1 | Required for meiotic nuclear division protein 1 homolog |
| | C6orf96 | |
| P31942 | HNRNPH3 | Heterogeneous nuclear ribonucleoprotein H3 (hnRNP |
| | HNRPH3 | H3) (Heterogeneous nuclear ribonucleoprotein 2H9) |
| | | (hnRNP 2H9) |
| Q9NZR2 | LRP1B | Low-density lipoprotein receptor-related protein 1B |
| | LRPDIT | (LRP-1B) (Low-density lipoprotein receptor-related |
| | | protein-deleted in tumor) (LRP-DIT) |
| Q16891 | IMMT | MICOS complex subunit MIC60 (Cell proliferation- |
| | HMP | inducing gene 4/52 protein) (Mitochondrial inner |
| | MIC60 | membrane protein) (Mitofilin) (p87/89) |
| | MINOS2 | |
| | PIG4 PIG52 | |
| A4D1N4 | CHCHD3 | MICOS complex subunit |
| | hCG_2014841 | |
| | tcag7.1158 | |
| Q9NX63 | CHCHD3 | MICOS complex subunit MIC19 (Coiled-coil-helix- |
| | MIC19 | coiled-coil-helix domain-containing protein 3) |
| | MINOS3 | |
| Q6NTF9 | RHBDD2 | Rhomboid domain-containing protein 2 |
| | RHBDL7 | |
| Q6P1M9 | ARMCX5 | Armadillo repeat-containing X-linked protein 5 |
| O00148 | DDX39A | ATP-dependent RNA helicase DDX39A (EC 3.6.4.13) |
| | DDX39 | (DEAD box protein 39) (Nuclear RNA helicase URH49) |
| Q6UY01 | LRRC31 | Leucine-rich repeat-containing protein 31 |
| | UNQ9367/ | |
| | PRO34156 | |
| Q8IYT3 | CCDC170 | Coiled-coil domain-containing protein 170 |
| | C6orf97 | |
| Q2L6I2 | ABCF1 | ABC50 protein (ATP-binding cassette, sub-family F |
| | ABC50 | (GCN20), member 1) (ATP-binding cassette, sub-family |
| | hCG_26012 | F (GCN20), member 1, isoform CRA_a) |
| Q8NE71 | ABCF1 | ATP-binding cassette sub-family F member 1 (ATP- |
| | ABC50 | binding cassette 50) (TNF-alpha-stimulated ABC |
| | | protein) |
| Q99459 | CDC5L | Cell division cycle 5-like protein (Cdc5-like protein) |
| | KIAA0432 | (Pombe cdc5-related protein) |
| | PCDC5RP | |
| P35580 | MYH10 | Myosin-10 (Cellular myosin heavy chain, type B) |
| | | (Myosin heavy chain 10) (Myosin heavy chain, non- |
| | | muscle IIb) (Non-muscle myosin heavy chain B) |
| | | (NMMHC-B) (Non-muscle myosin heavy chain IIb) |
| | | (NMMHC II-b) (NMMHC-IIB) |
| P50914 | RPL14 | 60S ribosomal protein L14 (CAG-ISL 7) (Large |
| | | ribosomal subunit protein eL14) |
| Q9C093 | SPEF2 | Sperm flagellar protein 2 (Protein KPL2) |
| | KIAA1770 | |
| | KPL2 | |
| P08729 | KRT7 SCL | Keratin, type II cytoskeletal 7 (Cytokeratin-7) (CK-7) |
| | | (Keratin-7) (K7) (Sarcolectin) (Type-II keratin Kb7) |
| Q9BTQ7 | | Similar to ribosomal protein L23 (Fragment) |
| Q96RT7 | TUBGCP6 | Gamma-tubulin complex component 6 (GCP-6) |
| | GCP6 | |
| | KIAA1669 | |
| Q5M8Q0 | Rpl15 | Ribosomal protein L15 |
| | mCG_10029 | |
| Q9CZM2 | Rpl15 | 60S ribosomal protein L15 |
| Q9BS75 | KLHL20 | KLHL20 protein (Kelch-like 20 (Drosophila), isoform |
| | hCG_23698 | CRA_a) |
| P82970 | HMGN5 | High mobility group nucleosome-binding domain- |
| | NSBP1 | containing protein 5 (Nucleosome-binding protein 1) |
| A0A024QZW2 | NOL7 | Nucleolar protein 7, 27 kDa, isoform CRA_a |
| | hCG_37417 | |
| Q9UMY1 | NOL7 | Nucleolar protein 7 (Nucleolar protein of 27 kDa) |
| | C6orf90 | |
| | NOP27 | |
| P62907 | Rpl10a | 60S ribosomal protein L10a |
| P78527 | PRKDC | DNA-dependent protein kinase catalytic subunit (DNA- |
| | HYRC | PK catalytic subunit) (DNA-PKcs) (EC 2.7.11.1) |
| | HYRC1 | (DNPK1) (p460) |
| B4E1W3 | | cDNA FLJ51732, highly similar to Peroxisomal NADH |
| | | pyrophosphatase NUDT12 (EC 3.6.1.22) |
| Q9BQG2 | NUDT12 | Peroxisomal NADH pyrophosphatase NUDT12 (EC |
| | | 3.6.1.22) (Nucleoside diphosphate-linked moiety X motif |
| | | 12) (Nudix motif 12) |
| P46779 | RPL28 | 60S ribosomal protein L28 (Large ribosomal subunit |
| | | protein eL28) |
| P22626 | HNRNPA2B1 | Heterogeneous nuclear ribonucleoproteins A2/B1 |
| | HNRPA2B1 | (hnRNP A2/B1) |
| Q96Q15 | SMG1 ATX | Serine/threonine-protein kinase SMG1 (SMG-1) (hSMG- |
| | KIAA0421 | 1) (EC 2.7.11.1) (61E3.4) (Lambda/iota protein kinase C- |
| | LIP | interacting protein) (Lambda-interacting protein) |
| A0A024R4M0 | RPS9 | 40S ribosomal protein S9 (Ribosomal protein S9, |
| | hCG_2009111 | isoform CRA_a) |
| P46781 | RPS9 | 40S ribosomal protein S9 (Small ribosomal subunit |
| | | protein uS4) |
| Q96T23 | RSF1 | Remodeling and spacing factor 1 (Rsf-1) (HBV pX- |
| | HBXAP | associated protein 8) (Hepatitis B virus X-associated |
| | XAP8 | protein) (p325 subunit of RSF chromatin-remodeling |
| | | complex) |
| P60709 | ACTB | Actin, cytoplasmic 1 (Beta-actin) [Cleaved into: Actin, |
| | | cytoplasmic 1,N-terminally processed] |
| Q96RL1 | UIMC1 | BRCA1-A complex subunit RAP80 (Receptor-associated |
| | RAP80 | protein 80) (Retinoid X receptor-interacting protein 110) |
| | RXRIP110 | (Ubiquitin interaction motif-containing protein 1) |
| Q96A11 | GAL3ST3 | Galactose-3-O-sulfotransferase 3 (Gal3ST-3) (EC 2.8.2.—) |
| | | (Beta-galactose-3-O-sulfotransferase 3) (Gal3ST3) |
| | | (Gal-beta-1, 3-GalNAc 3′-sulfotransferase 3) |
| P62847 | RPS24 | 40S ribosomal protein S24 (Small ribosomal subunit |
| | | protein eS24) |
| Q9NSI6 | BRWD1 | Bromodomain and WD repeat-containing protein 1 (WD |
| | C21orf107 | repeat-containing protein 9) |
| | WDR9 | |
| A0A024R1X8 | JUP | Junction plakoglobin, isoform CRA_a |
| | hCG_1771506 | |
| Q96QZ7 | MAGI1 | Membrane-associated guanylate kinase, WW and PDZ |
| | AIP3 | domain-containing protein 1 (Atrophin-1-interacting |
| | BAIAP1 | protein 3) (AIP-3) (BAI1-associated protein 1) (BAP-1) |
| | BAP1 | (Membrane-associated guanylate kinase inverted 1) |
| | TNRC19 | (MAGI-1) (Trinucleotide repeat-containing gene 19 |
| | | protein) (WW domain-containing protein 3) (WWP3) |
| A8K4C8 | RPL13 | 60S ribosomal protein L13 |
| | hCG_1723872 | |
| P26373 | RPL13 | 60S ribosomal protein L13 (Breast basic conserved |
| | BBC1 | protein 1) (Large ribosomal subunit protein eL13) |
| | OK/SW- | |
| | cl.46 | |
| P46019 | PHKA2 | Phosphorylase b kinase regulatory subunit alpha, liver |
| | PHKLA | isoform (Phosphorylase kinase alpha L subunit) |
| | PYK | |
| O60506 | SYNCRIP | Heterogeneous nuclear ribonucleoprotein Q (hnRNP Q) |
| | HNRPQ | (Glycine- and tyrosine-rich RNA-binding protein) (GRY- |
| | NSAP1 | RBP) (NS1-associated protein 1) (Synaptotagmin- |
| | | binding, cytoplasmic RNA-interacting protein) |
| Q96Q42 | ALS2 | Alsin (Amyotrophic lateral sclerosis 2 chromosomal |
| | ALS2CR6 | region candidate gene 6 protein) (Amyotrophic lateral |
| | KIAA1563 | sclerosis 2 protein) |
| Q8IYJ3 | SYTL1 | Synaptotagmin-like protein 1 (Exophilin-7) (Protein |
| | SLP1 | JFC1) |
| | SB146 | |
| A0A024RDH8 | RPL34 | Ribosomal protein L34, isoform CRA_a |
| | hCG_2027853 | |
| P49207 | RPL34 | 60S ribosomal protein L34 (Large ribosomal subunit |
| | | protein eL34) |
| Q9P2M7 | CGN | Cingulin |
| | KIAA1319 | |
| Q96BT3 | CENPT | Centromere protein T (CENP-T) (Interphase centromere |
| | C16orf56 | complex protein 22) |
| | ICEN22 | |
| Q0VF96 | CGNL1 | Cingulin-like protein 1 (Junction-associated coiled-coil |
| | JACOP | protein) (Paracingulin) |
| | KIAA1749 | |
| Q96M95 | CCDC42 | Coiled-coil domain-containing protein 42 |
| | CCDC42A | |
| P52597 | HNRNPF | Heterogeneous nuclear ribonucleoprotein F (hnRNP F) |
| | HNRPF | (Nucleolin-like protein mcs94-1) [Cleaved into: |
| | | Heterogeneous nuclear ribonucleoprotein F, N-terminally |
| | | processed] |
| O96008 | TOMM40 | Mitochondrial import receptor subunit TOM40 homolog |
| | C19orf1 | (Protein Haymaker) (Translocase of outer membrane 40 |
| | PEREC1 | kDa subunit homolog) (p38.5) |
| | TOM40 | |
| Q96BS4 | FBL | FBL protein (Putative uncharacterized protein) |
| | | (Fragment) |
| Q9H501 | ESF1 | ESF1 homolog (ABT1-associated protein) |
| | ABTAP | |
| | C20orf6 | |
| | HDCMC28P | |
| Q6PHZ2 | Camk2d | Calcium/calmodulin-dependent protein kinase type II |
| | Kiaa4163 | subunit delta (CaM kinase II subunit delta) (CaMK-II |
| | | subunit delta) (EC 2.7.11.17) |
| Q07020 | RPL18 | 60S ribosomal protein L18 (Large ribosomal subunit |
| | | protein eL18) |
| Q8TF72 | SHROOM3 | Protein Shroom3 (Shroom-related protein) (hShrmL) |
| | KIAA1481 | |
| | SHRML | |
| | MSTP013 | |
| Q8TE73 | DNAH5 | Dynein heavy chain 5, axonemal (Axonemal beta dynein |
| | DNAHC5 | heavy chain 5) (Ciliary dynein heavy chain 5) |
| | HL1 | |
| | KIAA1603 | |
| O75475 | PSIP1 | PC4 and SFRS1-interacting protein (CLL-associated |
| | DFS70 | antigen KW-7) (Dense fine speckles 70 kDa protein) |
| | LEDGF | (DPS 70) (Lens epithelium-derived growth factor) |
| | PSIP2 | (Transcriptional coactivator p75/p52) |
| E9KL44 | | Epididymis tissue sperm binding protein Li 14m |
| P40939 | HADHA | Trifunctional enzyme subunit alpha, mitochondrial (78 |
| | HADH | kDa gastrin-binding protein) (TP-alpha) [Includes: Long- |
| | | chain enoyl-CoA hydratase (EC 4.2.1.17); Long chain 3- |
| | | hydroxyacyl-CoA dehydrogenase (EC 1.1.1.211)] |
| Q9HB09 | BCL2L12 | Bcl-2-like protein 12 (Bcl2-L-12) (Bcl-2-related proline- |
| | BPR | rich protein) |
| O75367 | H2AFY | Core histone macro-H2A.1 (Histone macroH2A1) |
| | MACROH2A1 | (mH2A1) (Histone H2A.y) (H2A/y) (Medulloblastoma |
| | | antigen MU-MB-50.205) |
| Q8N6Z2 | MTRF1 | MTRF1 protein (Mitochondrial translational release |
| | hCG_32761 | factor 1, isoform CRA_b) (Peptide chain release factor 1, |
| | | mitochondrial) |
| Q8TCU4 | ALMS1 | Alstrom syndrome protein 1 |
| | KIAA0328 | |
| A0JNW5 | UHRF1BP1L | UHRF1-binding protein 1-like |
| | KIAA0701 | |
| O75643 | SNRNP200 | U5 small nuclear ribonucleoprotein 200 kDa helicase |
| | ASCC3L1 | (EC 3.6.4.13) (Activating signal cointegrator 1 complex |
| | HELIC2 | subunit 3-like 1) (BRR2 homolog) (U5 snRNP-specific |
| | KIAA0788 | 200 kDa protein) (U5-200KD) |
| A7E2E1 | SMARCA4 | SWI/SNF related, matrix associated, actin dependent |
| | hCG_29955 | regulator of chromatin, subfamily a, member 4 |
| | | (SWI/SNF related, matrix associated, actin dependent |
| | | regulator of chromatin, subfamily a, member 4, isoform |
| | | CRA_a) (cDNA FLJ77531, highly similar to Homo sapiens |
| | | SWI/SNF related, matrix associated, actin |
| | | dependent regulator of chromatin, subfamily a, member 4 |
| | | (SMARCA4), mRNA) |
| P51532 | SMARCA4 | Transcription activator BRG1 (EC 3.6.4.—) (ATP- |
| | BAF190A | dependent helicase SMARCA4) (BRG1-associated factor |
| | BRG1 | 190A) (BAF190A) (Mitotic growth and transcription |
| | SNF2B | activator) (Protein BRG-1) (Protein brahma homolog 1) |
| | SNF2L4 | (SNF2-beta) (SWI/SNF-related matrix-associated actin- |
| | | dependent regulator of chromatin subfamily A member |
| | | 4) |
| O00418 | EEF2K | Eukaryotic elongation factor 2 kinase (eEF-2 kinase) |
| | | (eEF-2K) (EC 2.7.11.20) (Calcium/calmodulin- |
| | | dependent eukaryotic elongation factor 2 kinase) |
| Q96CN4 | EVI5L | EVI5-like protein (Ecotropic viral integration site 5-like |
| | | protein) |
| Q9H8V3 | ECT2 | Protein ECT2 (Epithelial cell-transforming sequence 2 |
| | | oncogene) |
| Q5T3F8 | TMEM63B | CSC1-like protein 2 (Transmembrane protein 63B) |
| | C6orf110 | |
| Q8NAJ6 | | cDNA FLJ35251 fis, clone PROST2003635, weakly |
| | | similar to MULTIFUNCTIONAL AMINOACYL-TRNA |
| | | SYNTHETASE |
| A0A0C4DG40 | SYNE1 | Nesprin-1 |
| Q8NF91 | SYNE1 | Nesprin-1 (Enaptin) (KASH domain-containing protein |
| | C6orf98 | 1) (KASH1) (Myocyte nuclear envelope protein 1) |
| | KIAA0796 | (Myne-1) (Nuclear envelope spectrin repeat protein 1) |
| | KIAA1262 | (Synaptic nuclear envelope protein 1) (Syne-1) |
| | KIAA1756 | |
| | MYNE1 | |
| Q8TDI0 | CHD5 | Chromodomain-helicase-DNA-binding protein 5 (CHD- |
| | KIAA0444 | 5) (EC 3.6.4.12) (ATP-dependent helicase CHD5) |
| Q9NU22 | MDN1 | Midasin (MIDAS-containing protein) |
| | KIAA0301 | |
| Q8WXH0 | SYNE2 | Nesprin-2 (KASH domain-containing protein 2) |
| | KIAA1011 | (KASH2) (Nuclear envelope spectrin repeat protein 2) |
| | NUA | (Nucleus and actin connecting element protein) (Protein |
| | | NUANCE) (Synaptic nuclear envelope protein 2) (Syne- |
| | | 2) |
| Q9Y277 | VDAC3 | Voltage-dependent anion-selective channel protein 3 |
| | | (VDAC-3) (hVDAC3) (Outer mitochondrial membrane |
| | | protein porin 3) |
| Q96QE3 | ATAD5 | ATPase family AAA domain-containing protein 5 |
| | C17orf41 | (Chromosome fragility-associated gene 1 protein) |
| | FRAG1 | |
| Q9BXJ9 | NAA15 | N-alpha-acetyltransferase 15, NatA auxiliary subunit |
| | GA19 | (Gastric cancer antigen Ga19) (N-terminal |
| | NARG1 | acetyltransferase) (NMDA receptor-regulated protein 1) |
| | NATH | (Protein tubedown-1) (Tbdn100) |
| | TBDN100 | |
| Q8IUE6 | HIST2H2AB | Histone H2A type 2-B |
| Q5TZA2 | CROCC | Rootletin (Ciliary rootlet coiled-coil protein) |
| | KIAA0445 | |
| A0A024RAS2 | H2AFJ | Histone H2A |
| | hCG_1639762 | |
| Q9BTM1 | H2AFJ | Histone H2A.J (H2a/j) |
| Q8NEN9 | PDZD8 | PDZ domain-containing protein 8 (Sarcoma antigen NY- |
| | PDZK8 | SAR-84/NY-SAR-104) |
| Q14683 | SMC1A | Structural maintenance of chromosomes protein 1A |
| | DXS423E | (SMC protein 1A) (SMC-1-alpha) (SMC-1A) (Sb1.8) |
| | KIAA0178 | |
| | SB1.8 | |
| | SMC1 | |
| | SMC1L1 | |
| Q68EN4 | SMC1A | SMC1A protein (Fragment) |
| Q7Z7G8 | VPS13B | Vacuolar protein sorting-associated protein 13B (Cohen |
| | CHS1 | syndrome protein 1) |
| | COH1 | |
| | KIAA0532 | |
| Q7Z7A1 | CNTRL | Centriolin (Centrosomal protein 1) (Centrosomal protein |
| | CEP1 | of 110 kDa) (Cep110) |
| | CEP110 | |
| O95613 | PCNT | Pericentrin (Kendrin) (Pericentrin-B) |
| | KIAA0402 | |
| | PCNT2 | |
| A0A140VK14 | | Testicular secretory protein Li 14 |
| P49448 | GLUD2 | Glutamate dehydrogenase 2, mitochondrial (GDH 2) (EC |
| | GLUDP1 | 1.4.1.3) |
| Q5VTT5 | MYOM3 | Myomesin-3 (Myomesin family member 3) |
| Q7Z612 | | Acidic ribosomal phosphoprotein P1 |
| O00567 | NOP56 | Nucleolar protein 56 (Nucleolar protein 5A) |
| | NOL5A | |
| Q9Y2X3 | NOP58 | Nucleolar protein 58 (Nucleolar protein 5) |
| | NOL5 | |
| | NOP5 | |
| | HSPC120 | |
| A0A0C4DFX4 | | Uncharacterized protein (Fragment) |
| Q6ZNL4 | FLJ00279 | FLJ00279 protein (Fragment) |
| Q6ZWK7 | | cDNA FLJ16045 fis, clone CTONG2000042, weakly |
| | | similar to ALPHA-2-MACROGLOBULIN |
| Q7Z388 | DPY19L4 | Probable C-mannosyltransferase DPY19L4 (EC 2.4.1.—) |
| | | (Dpy-19-like protein 4) (Protein dpy-19 homolog 4) |
| Q5T9S5 | CCDC18 | Coiled-coil domain-containing protein 18 (Sarcoma |
| | | antigen NY-SAR-24) |
| Q6ZV73 | FGD6 | FYVE, RhoGEF and PH domain-containing protein 6 |
| | KIAA1362 | (Zinc finger FYVE domain-containing protein 24) |
| | ZFYVE24 | |
| P25705 | ATP5A1 | ATP synthase subunit alpha, mitochondrial |
| | ATP5A | |
| | ATP5AL2 | |
| | ATPM | |
| P42285 | SKIV2L2 | Superkiller viralicidic activity 2-like 2 (EC 3.6.4.13) |
| | DOB1 | (ATP-dependent RNA helicase DOB1) (ATP-dependent |
| | KIAA0052 | RNA helicase SKIV2L2) (TRAMP-like complex |
| | Mtr4 | helicase) |
| Q00325 | SLC25A3 | Phosphate carrier protein, mitochondrial (Phosphate |
| | PHC | transport protein) (PTP) (Solute carrier family 25 |
| | OK/SW- | member 3) |
| | cl.48 | |
| P62753 | RPS6 | 40S ribosomal protein S6 (Phosphoprotein NP33) (Small |
| | OK/SW-cl.2 | ribosomal subunit protein eS6) |
| Q9BW34 | EEF1D | EEF1D protein (Fragment) |
| Q5K651 | SAMD9 | Sterile alpha motif domain-containing protein 9 (SAM |
| | C7orf5 | domain-containing protein 9) |
| | DRIF1 | |
| | KIAA2004 | |
| | OEF1 | |
| Q6W6M6 | | Antigen MLAA-44 |
| Q5T0F9 | CC2D1B | Coiled-coil and C2 domain-containing protein 1B (Five |
| | KIAA1836 | prime represser element under dual repression-binding |
| | | protein 2) (FRE under dual repression-binding protein 2) |
| | | (Freud-2) |
| P26641 | EEF1G | Elongation factor 1-gamma (EF-1-gamma) (eEF-1B |
| | EF1G | gamma) |
| | PRO1608 | |
| Q00839 | HNRNPU | Heterogeneous nuclear ribonucleoprotein U (hnRNP U) |
| | HNRPU | (Scaffold attachment factor A) (SAF-A) (p120) (pp120) |
| | SAFA | |
| | U21.1 | |
| Q9Y4C4 | MFHAS1 | Malignant fibrous histiocytoma-amplified sequence 1 |
| | MASL1 | (Malignant fibrous histiocytoma-amplified sequence with |
| | | leucine-rich tandem repeats 1) |
| P16050 | ALOX15 | Arachidonate 15-lipoxygenase (15-LOX) (15-LOX-1) |
| | LOG15 | (EC 1.13.11.33) (12/15-lipoxygenase) (Arachidonate 12- |
| | | lipoxygenase, leukocyte-type) (12-LOX) (EC 1.13.11.31) |
| | | (Arachidonate omega-6 lipoxygenase) |
| P16383 | GCFC2 | GC-rich sequence DNA-binding factor 2 (GC-rich |
| | C2orf3 GCF | sequence DNA-binding factor) (Transcription factor 9) |
| | TCF9 | (TCF-9) |
| P36578 | RPL4 RPL1 | 60S ribosomal protein L4 (60S ribosomal protein L1) |
| | | (Large ribosomal subunit protein uL4) |
| O76081 | RGS20 | Regulator of G-protein signaling 20 (RGS20) (Gz- |
| | RGSZ1 | selective GTPase-activating protein) (G(z)GAP) (Gz- |
| | ZGAP1 | GAP) (Regulator of G-protein signaling Z1) (Regulator |
| | | of Gz-selective protein signaling 1) |
| Q9Y6N9 | USH1C | Harmonin (Antigen NY-CO-38/NY-CO-37) |
| | AIE75 | (Autoimmune enteropathy-related antigen AIE-75) |
| | | (Protein PDZ-73) (Renal carcinoma antigen NY-REN-3) |
| | | (Usher syndrome type-1C protein) |
| Q15149 | PLEC | Plectin (PCN) (PLTN) (Hemidesmosomal protein 1) |
| | PLEC1 | (HD1) (Plectin-1) |
| O60333 | KIF1B | Kinesin-like protein KIF1B (Klp) |
| | KIAA0591 | |
| | KIAA1448 | |
| O60462 | NRP2 | Neuropilin-2 (Vascular endothelial cell growth factor |
| | VEGF165R2 | 165 receptor 2) |
| Q7Z3T9 | DKFZp686J | Neuropilin |
| | 1169 | |
| Q5THJ4 | VPS13D | Vacuolar protein sorting-associated protein 13D |
| | KIAA0453 | |
| Q9NRZ9 | HELLS | Lymphoid-specific helicase (EC 3.6.4.—) (Proliferation- |
| | PASG | associated SNF2-like protein) (SWI/SNF2-related |
| | SMARCA6 | matrix-associated actin-dependent regulator of chromatin |
| | Nbla10143 | subfamily A member 6) |
| Q96A08 | HIST1H2B | Histone H2B type 1-A (Histone H2B, testis) (TSH2B.1) |
| | A TSH2B | (hTSH2B) (Testis-specific histone H2B) |
| Q6UB99 | ANKRD11 | Ankyrin repeat domain-containing protein 11 (Ankyrin |
| | ANCO1 | repeat-containing cofactor 1) |
| I6L9F7 | HIST1H2BM | Histone H2B (Fragment) |
| P02538 | KRT6A | Keratin, type II cytoskeletal 6A (Cytokeratin-6A) (CK- |
| | K6A | 6A) (Cytokeratin-6D) (CK-6D) (Keratin-6A) (K6A) |
| | KRT6D | (Type-II keratin Kb6) (allergen Horn s 5) |
| Q6KC79 | NIPBL | Nipped-B-like protein (Delangin) (SCC2 homolog) |
| | IDN3 | |
| Q8NBU5 | ATAD1 | ATPase family AAA domain-containing protein 1 (EC |
| | FNP001 | 3.6.1.3) (Thorase) |
| E5KLM2 | | Mitochondrial dynamin-like 120 kDa protein |
| Q15772 | SPEG | Striated muscle preferentially expressed protein kinase |
| | APEG1 | (EC 2.7.11.1) (Aortic preferentially expressed protein 1) |
| | KIAA1297 | (APEG-1) |
| O14490 | DLGAP1 | Disks large-associated protein 1 (DAP-1) (Guanylate |
| | DAP1 | kinase-associated protein) (hGKAP) (PSD-95/SAP90- |
| | GKAP | binding protein 1) (SAP90/PSD-95-associated protein 1) |
| | | (SAPAP1) |
| Q5JSL3 | DOCK11 | Dedicator of cytokinesis protein 11 (Activated Cdc42- |
| | ZIZ2 | associated guanine nucleotide exchange factor) (ACG) |
| | | (Zizimin-2) |
| Q5VU43 | PDE4DIP | Myomegalin (Cardiomyopathy-associated protein 2) |
| | CMYA2 | (Phosphodiesterase 4D-interacting protein) |
| | KIAA0454 | |
| | KIAA0477 | |
| | MMGL | |
| Q658X5 | DKFZp666F1010 | Putative uncharacterized protein DKFZp666F1010 |
| | | (Fragment) |
| Q658W4 | DKFZp666M0710 | Putative uncharacterized protein DKFZp666M0710 |
| | | (Fragment) |
| Q63HR1 | DKFZp686P17171 | Putative uncharacterized protein DKFZp686P17171 |
| Q5VWT5 | ARAP | Activation-dependent, raft-recruited ADAP-like |
| | C1orf168 | phosphoprotein |
| Q92614 | MYO18A | Unconventional myosin-XVIIIa (Molecule associated |
| | CD245 | with JAK3 N-terminus) (MAJN) (Myosin containing a |
| | KIAA0216 | PDZ domain) (Surfactant protein receptor SP-R210) (SP- |
| | MYSPDZ | R210) |
| A0A024R4A0 | NCL | Nucleolin, isoform CRA_b |
| | hCG_33980 | |
| B3KM80 | NCL | Nucleolin, isoform CRA_c (cDNA FLJ10452 fis, clone |
| | hCG_33980 | NT2RP1000966, highly similar to NUCLEOLIN) |
| P19338 | NCL | Nucleolin (Protein C23) |
| P35527 | KRT9 | Keratin, type I cytoskeletal 9 (Cytokeratin-9) (CK-9) |
| | | (Keratin-9) (K9) |
| Q5T655 | CFAP58 | Cilia- and flagella-associated protein 58 (Coiled-coil |
| | C10orf80 | domain-containing protein 147) |
| | CCDC147 | |
| Q5TAX3 | ZCCHC11 | Terminal uridylyltransferase 4 (TUTase 4) (EC 2.7.7.52) |
| | KIAA0191 | (Zinc finger CCHC domain-containing protein 11) |
| | TUT4 | |
| Q9Y6I7 | WSB1 | WD repeat and SOCS box-containing protein 1 (WSB-1) |
| | SWIP1 | (SOCS box-containing WD protein SWiP-1) |
| Q9HC77 | CENPJ | Centromere protein J (CENP-J) (Centrosomal P4.1- |
| | CPAP LAP | associated protein) (LAG-3-associated protein) (LYST- |
| | LIP1 | interacting protein 1) |
| Q5H8C1 | FREM1 | FRAS1-related extracellular matrix protein 1 (Protein |
| | C9orf143 | QBRICK) |
| | C9orf145 | |
| | C9orf154 | |
| Q8N5G2 | TMEM57 | Macoilin (Transmembrane protein 57) |
| Q58F05 | NARG1 | NARG1 protein (Fragment) |
| Q59HE3 | | Calpastatin isoform a variant (Fragment) |
| Q59GX9 | | Ribosomal protein L5 variant (Fragment) |
| Q59FF1 | | Insulin-like growth factor binding protein 2 variant |
| | | (Fragment) |
| O75116 | ROCK2 | Rho-associated protein kinase 2 (EC 2.7.11.1) (Rho |
| | KIAA0619 | kinase 2) (Rho-associated, coiled-coil-containing protein |
| | | kinase 2) (Rho-associated, coiled-coil-containing protein |
| | | kinase II) (ROCK-II) (p164 ROCK-2) |
| Q53HW2 | | 60S acidic ribosomal protein P0 (Fragment) |
| Q53HR5 | | Elongation factor 1-alpha (Fragment) |
| P14136 | GFAP | Glial fibrillary acidic protein (GFAP) |
| Q562R1 | ACTBL2 | Beta-actin-like protein 2 (Kappa-actin) |
| A0A024R2G2 | FANCD2 | Fanconi anemia, complementation group D2, isoform |
| | hCG_1811443 | CRA_b |
| Q9BXW9 | FANCD2 | Fanconi anemia group D2 protein (Protein FACD2) |
| | FACD | |
| Q86XH1 | IQCA1 | IQ and AAA domain-containing protein 1 |
| | IQCA | |
| A1XBS5 | FAM92A | Protein FAM92A |
| | FAM92A1 | |
| Q9P273 | TENM3 | Teneurin-3 (Ten-3) (Protein Odd Oz/ten-m homolog 3) |
| | KIAA1455 | (Tenascin-M3) (Ten-m3) (Teneurin transmembrane |
| | ODZ3 | protein 3) |
| | TNM3 | |
| Q9P2K1 | CC2D2A | Coiled-coil and C2 domain-containing protein 2A |
| | KIAA1345 | |
| Q96BT1 | C3orf49 | Putative uncharacterized protein C3orf49 |
| P09651 | HNRNPA1 | Heterogeneous nuclear ribonucleoprotein A1 (hnRNP |
| | HNRPA1 | A1) (Helix-destabilizing protein) (Single-strand RNA- |
| | | binding protein) (hnRNP core protein A1) [Cleaved into: |
| | | Heterogeneous nuclear ribonucleoprotein A1, N- |
| | | terminally processed] |
| Q9P225 | DNAH2 | Dynein heavy chain 2, axonemal (Axonemal beta dynein |
| | DNAHC2 | heavy chain 2) (Ciliary dynein heavy chain 2) (Dynein |
| | DNHD3 | heavy chain domain-containing protein 3) |
| | KIAA1503 | |
| Q4KM60 | Rpl10a | Ribosomal protein (Fragment) |
| | Serpina6 | |
| Q32Q62 | RSL1D1 | RSL1D1 protein (Fragment) |
| Q9H611 | PIF1 | ATP-dependent DNA helicase PIF1 (EC 3.6.4.12) (DNA |
| | C15orf20 | repair and recombination helicase PIF1) (PIF1/RRM3 |
| | | DNA helicase-like protein) |
| Q86Y46 | KRT73 | Keratin, type II cytoskeletal 73 (Cytokeratin-73) (CK-73) |
| | K6IRS3 | (Keratin-73) (K73) (Type II inner root sheath-specific |
| | KB36 | keratin-K6irs3) (Type-II keratin Kb36) |
| | KRT6IRS3 | |
| Q0QEN7 | ATP5B | ATP synthase subunit beta (EC 3.6.3.14) (Fragment) |
| B3KU66 | | cDNA FLJ39263 fis, clone OCBBF2009571, highly |
| | | similar to ATP-dependent RNA helicase A (EC 3.6.1.—) |
| Q08211 | DHX9 | ATP-dependent RNA helicase A (RHA) (EC 3.6.4.13) |
| | DDX9 LKP | (DEAH box protein 9) (Leukophysin) (LKP) (Nuclear |
| | NDH2 | DNA helicase II) (NDH II) |
| O15078 | CEP290 | Centrosomal protein of 290 kDa (Cep290) (Bardet-Biedl |
| | BBS14 | syndrome 14 protein) (Cancer/testis antigen 87) (CT87) |
| | KIAA0373 | (Nephrocystin-6) (Tumor antigen se2-2) |
| | NPHP6 | |
| Q05BJ6 | CEP290 | CEP290 protein |
| Q92538 | GBF1 | Golgi-specific brefeldin A-resistance guanine nucleotide |
| | KIAA0248 | exchange factor 1 (BFA-resistant GEF 1) |
| Q4G0J3 | LARP7 | La-related protein 7 (La ribonucleoprotein domain family |
| | HDCMA18P | member 7) (P-TEFb-interaction protein for 7SK stability) |
| | | (PIP7S) |
| Q15397 | PUM3 | Pumilio homolog 3 (HBV X-transactivated gene 5 |
| | cPERP-C | protein) (HBV XAg-transactivated protein 5) (Minor |
| | KIAA0020 | histocompatibility antigen HA-8) (HLA-HA8) |
| | PUF-A | |
| | XTP5 | |
| Q7RTY7 | OVCH1 | Ovochymase-1 (EC 3.4.21.—) |
| Q5SPB7 | ino80 | INO80 complex subunit |
| | si:ch211- | |
| | 244p18.3 | |
| Q9Y3V2 | RWDD3 | RWD domain-containing protein 3 (RWD domain- |
| | RSUME | containing sumoylation enhancer) (RSUME) |
| Q9HCR9 | PDE11A | Dual 3′,5′-cyclic-AMP and -GMP phosphodiesterase 11A |
| | | (EC 3.1.4.35) (EC 3.1.4.53) (cAMP and cGMP |
| | | phosphodiesterase 11A) |
| Q9NR48 | ASH1L | Histone-lysine N-methyltransferase ASH1L (EC |
| | KIAA1420 | 2.1.1.43) (ASH1-like protein) (huASH1) (Absent small |
| | KMT2H | and homeotic disks protein 1 homolog) (Lysine N- |
| | | methyltransferase 2H) |
| Q09428 | ABCC8 | ATP-binding cassette sub-family C member 8 |
| | HRINS | (Sulfonylurea receptor 1) |
| | SUR SUR1 | |
| Q5JU67 | CFAP157 | Cilia- and flagella-associated protein 157 |
| | C9orf117 | |
| D3DR32 | MPHOSPH1 | M-phase phosphoprotein 1, isoform CRA_a |
| | hCG_23744 | |
| G5E9G0 | RPL3 ASC-1 | 60S ribosomal protein L3 (Ribosomal protein L3, |
| | hCG_2015191 | isoform CRA_e) |
| D3DS91 | AKAP6 | A kinase (PRKA) anchor protein 6, isoform CRA_b |
| | hCG_1812123 | |
| A0A0A7M1X5 | LMNB2 | Lamin B2, isoform CRA_b (Lamin B3) |
| | hCG_2004338 | |
| A0A024R5M9 | NUMA1 | Nuclear mitotic apparatus protein 1, isoform CRA_a |
| | hCG_2017131 | |
| Q4G0X9 | CCDC40 | Coiled-coil domain-containing protein 40 |
| | KIAA1640 | |
| D3DTT5 | TBKBP1 | TBK1 binding protein 1, isoform CRA_a |
| | hCG_1813987 | |
| G5E972 | TMPO | Lamina-associated polypeptide 2, isoforms beta/gamma |
| | hCG_2015322 | (Thymopoietin, isoform CRA_d) |
| D6W5D1 | KIAA1212 | KIAA1212, isoform CRA_a |
| | hCG_1817741 | |
| U3KQK0 | HIST1H2BN | Histone H2B |
| | hCG_1743059 | |
| D6RGI3 | SEPT11 | Septin 11, isoform CRA_b (Septin-11) |
| | hCG_24410 | |
| B4DDB6 | HNRPA3 | Heterogeneous nuclear ribonucleoprotein A3, isoform |
| | hCG_2005824 | CRA_a (cDNA FLJ52659, highly similar to |
| | | Heterogeneous nuclear ribonucleoprotein A3) (cDNA, |
| | | FLJ79333, highly similar to Heterogeneous nuclear |
| | | ribonucleoprotein A3) |
| Q8TE76 | MORC4 | MORC family CW-type zinc finger protein 4 (Zinc |
| | ZCW4 | finger CW-type coiled-coil domain protein 2) (Zinc |
| | ZCWCC2 | finger CW-type domain protein 4) |
| Q8NCM8 | DYNC2H1 | Cytoplasmic dynein 2 heavy chain 1 (Cytoplasmic |
| | DHC1B | dynein 2 heavy chain) (Dynein Cytoplasmic heavy chain |
| | DHC2 | 2) (Dynein heavy chain 11) (hDHC11) (Dynein heavy |
| | DNCH2 | chain isotype 1B) |
| | DYH1B | |
| | KIAA1997 | |
| Q6PIF6 | MYO7B | Unconventional myosin-VIIb |
| Q8NB66 | UNC13C | Protein unc-13 homolog C (Munc13-3) |
| A0A1U9X7W7 | | HSPA1L |
| P34931 | HSPA1L | Heat shock 70 kDa protein 1-like (Heat shock 70 kDa |
| | | protein 1L) (Heat shock 70 kDa protein 1-Hom) (HSP70- |
| | | Hom) |
| A4D0S4 | LAMB4 | Laminin subunit beta-4 (Laminin beta-1-related protein) |
| Q8N309 | LRRC43 | Leucine-rich repeat-containing protein 43 |
| Q8TDW7 | FAT3 | Protocadherin Fat 3 (hFat3) (Cadherin family member |
| | CDHF15 | 15) (FAT tumor suppressor homolog 3) |
| | KIAA1989 | |
| A5WVL9 | dapk1 | Death-associated protein kinase (Death-associated |
| | si:ch211- | protein kinase 1) |
| | 66i11.1 | |
| P05141 | SLC25A5 | ADP/ATP translocase 2 (ADP, ATP carrier protein 2) |
| | ANT2 | (ADP, ATP carrier protein, fibroblast isoform) (Adenine |
| | | nucleotide translocate 2) (ANT 2) (Solute carrier family |
| | | 25 member 5) [Cleaved into: ADP/ATP translocase 2, N- |
| | | terminally processed] |
| Q6NVC0 | SLC25A5 | SLC25A5 protein (Fragment) |
| P12236 | SLC25A6 | ADP/ATP translocase 3 (ADP, ATP carrier protein 3) |
| | ANT3 | (ADP, ATP carrier protein, isoform T2) (ANT 2) |
| | CDABP0051 | (Adenine nucleotide translocator 3) (ANT 3) (Solute |
| | | carrier family 25 member 6) [Cleaved into: ADP/ATP |
| | | translocase 3, N-terminally processed] |
| Q6I9V5 | SLC25A6 | SLC25A6 protein (Solute carrier family 25 |
| | hCG_1746794 | (Mitochondrial carrier adenine nucleotide translocator), |
| | | member 6) (cDNA, FLJ92654, highly similar to Homo sapiens |
| | | solute carrier family 25 (mitochondrial carrier; |
| | | adenine nucleotide translocator), member 6 (SLC25A6), |
| | | mRNA) |
| Q0VGD6 | HNRPR | HNRPR protein (Fragment) |
| A0A024R3T8 | PARP1 | Poly [ADP-ribose] polymerase (PARP) (EC 2.4.2.30) |
| | hCG_14746 | |
| P09874 | PARP1 | Poly [ADP-ribose] polymerase 1 (PARP-1) (EC 2.4.2.30) |
| | ADPRT | (ADP-ribosyltransferase diphtheria toxin-like 1) |
| | PPOL | (ARTD1) (NAD(+) ADP-ribosyltransferase 1) (ADPRT |
| | | 1) (Poly[ADP-ribose] synthase 1) |
| Q8IVF2 | AHNAK2 | Protein AHNAK2 |
| | C14orf78 | |
| | KIAA2019 | |
| Q9BQG0 | MYBBP1A | Myb-binding protein 1A |
| | P160 | |
| A6PVS8 | LRRIQ3 | Leucine-rich repeat and IQ domain-containing protein 3 |
| | LRRC44 | (Leucine-rich repeat-containing protein 44) |
| A8K6K6 | | cDNA FLJ76880 |
| A8K2G7 | | cDNA FLJ76071, highly similar to Homo sapiens |
| | | filamin A interacting protein 1 (FILIP1), mRNA |
| B0AZQ4 | | Structural maintenance of chromosomes protein |
| Q9P1Z9 | CCDC180 | Coiled-coil domain-containing protein 180 |
| | C9orf174 | |
| | KIAA1529 | |
| Q9UFH2 | DNAH17 | Dynein heavy chain 17, axonemal (Axonemal beta |
| | DNAHL1 | dynein heavy chain 17) (Axonemal dynein heavy chain- |
| | DNEL2 | like protein 1) (Ciliary dynein heavy chain 17) (Ciliary |
| | | dynein heavy chain-like protein 1) (Dynein light chain 2, |
| | | axonemal) |
| B2R5B3 | | Histone H2A |
| B2RAM8 | | cDNA, FLJ95007, highly similar to Homo sapiens |
| | | BRCA1 associated RING domain 1 (BARD1), mRNA |
| Q68CZ1 | RPGRIP1L | Protein fantom (Nephrocystin-8) (RPGR-interacting |
| | FTM | protein 1-like protein) (RPGRIP1-like protein) |
| | KIAA1005 | |
| | NPHP8 | |
| Q2QL34 | MPV17L | Mpv17-like protein (M-LP homolog) (M-LPH) |
| Q13948 | CUX1 | Protein CASP |
| | CUTL1 | |
| B3KX72 | | cDNA FLJ44920 fis, clone BRAMY3011501, highly |
| | | similar to Heterogeneous nuclear ribonucleoprotein U |
| Q9NVI7 | ATAD3A | ATPase family AAA domain-containing protein 3A |
| B3KS36 | | cDNA FLJ35376 fis, clone SKMUS2004044, highly |
| | | similar to Homo sapiens ribosomal protein L3 (RPL3), |
| | | transcript variant 2, mRNA |
| D7EZH4 | | SNF2LT |
| Q9C0G6 | DNAH6 | Dynein heavy chain 6, axonemal (Axonemal beta dynein |
| | DNAHC6 | heavy chain 6) (Ciliary dynein heavy chain 6) |
| | DNHL1 | |
| | HL2 | |
| | KIAA1697 | |
| O60524 | NEMF | Nuclear export mediator factor NEMF (Antigen NY-CO- |
| | SDCCAG1 | 1) (Serologically defined colon cancer antigen 1) |
| B4DWU6 | | cDNA FLJ51361, highly similar to Keratin, type II |
| | | cytoskeletal 6A |
| B4DXG0 | | cDNA FLJ57651, highly similar to Ketosamine-3-kinase |
| | | (EC 2.7.1.—) |
| B4DGN6 | | cDNA FLJ50007 |
| B4DXQ8 | | cDNA FLJ52940, highly similar to Mortality factor 4- |
| | | like protein 2 |
| Q7L099 | RUFY3 | Protein RUFY3 (RUN and FYVE domain-containing |
| | KIAA0871 | protein 3) (Rap2-interacting protein x) (RIPx) (Single |
| | | axon-regulated protein) (Singar) |
| Q9C099 | LRRCC1 | Leucine-rich repeat and coiled-coil domain-containing |
| | CLERC | protein 1 (Centrosomal leucine-rich repeat and coiled- |
| | KIAA1764 | coil domain-containing protein) |
| B4DYY8 | | cDNA FLJ60374 |
| Q14439 | GPR176 | G-protein coupled receptor 176 (HB-954) |
| B4DZM3 | | cDNA FLJ61500, highly similar to NNP-1 protein |
| P62318 | SNRPD3 | Small nuclear ribonucleoprotein Sm D3 (Sm-D3) |
| | | (snRNP core protein D3) |
| B4E1T1 | | cDNA FLJ54081, highly similar to Keratin, type II |
| | | cytoskeletal 5 |
| B4DLB1 | | cDNA FLJ58017, moderately similar to Treacle protein |
| Q8TC59 | PIWIL2 | Piwi-like protein 2 (EC 3.1.26.—) (Cancer/testis antigen |
| | HILI | 80) (CT80) |
| Q16513 | PKN2 | Serine/threonine-protein kinase N2 (EC 2.7.11.13) (PKN |
| | PRK2 | gamma) (Protein kinase C-like 2) (Protein-kinase C- |
| | PRKCL2 | related kinase 2) |
| O75923 | DYSF | Dysferlin (Dystrophy-associated fer-1-like protein) (Fer- |
| | FER1L1 | 1-like protein 1) |
| Q5RF89 | DKFZp469 | Putative uncharacterized protein DKFZp469P0721 |
| | P0721 | |
| Q9UBN4 | TRPC4 | Short transient receptor potential channel 4 (TrpC4) |
| | | (Trp-related protein 4) (hTrp-4) (hTrp4) |
| P62826 | RAN | GTP-binding nuclear protein Ran (Androgen receptor- |
| | ARA24 | associated protein 24) (GTPase Ran) (Ras-like protein |
| | OK/SW- | TC4) (Ras-related nuclear protein) |
| | cl.81 | |
| Q6NTA2 | HNRNPL | HNRNPL protein (Fragment) |
| B4DPC0 | | cDNA FLJ52713, moderately similar to Mus musculus |
| | | leucine rich repeat (in FLII) interacting protein 1 |
| | | (Lrrfip1), mRNA |
| B7Z2C5 | | cDNA FLJ50492, highly similar to Cyclin-dependent |
| | | kinase-like 3 (EC 2.7.11.22) |
| Q86TI0 | TBC1D1 | TBC1 domain family member 1 |
| | KIAA1108 | |
| Q15233 | NONO | Non-POU domain-containing octamer-binding protein |
| | NRB54 | (NonO protein) (54 kDa nuclear RNA- and DNA-binding |
| | | protein) (55 kDa nuclear protein) (DNA-binding |
| | | p52/p100 complex, 52 kDa subunit) (NMT55) (p54(nrb)) |
| | | (p54nrb) |
| B7Z4E3 | RPL31 | 60S ribosomal protein L31 (cDNA FLJ58908, highly |
| | | similar to 60S ribosomal protein L31) |
| B7Z7K9 | | CDNA FLJ51382 |
| Q92833 | JARID2 | Protein Jumonji (Jumonji/ARID domain-containing |
| | JMJ | protein 2) |
| Q8N398 | VWA5B2 | von Willebrand factor A domain-containing protein 5B2 |
| Q9BVH8 | VWA5B2 | VWA5B2 protein (Fragment) |
| Q6ZU80 | CEP128 | Centrosomal protein of 128 kDa (Cep128) |
| | C14orf145 | |
| | C14orf61 | |
| P46013 | MKI67 | Proliferation marker protein Ki-67 (Antigen identified by |
| | | monoclonal antibody Ki-67) (Antigen KI-67) (Antigen |
| | | Ki67) |
| A2A547 | Rpl19 | Ribosomal protein L19 |
| E4W6B6 | RPL27 | RPL27/NME2 fusion protein (Fragment) |
| O15050 | TRANK1 | TPR and ankyrin repeat-containing protein 1 (Lupus |
| | KIAA0342 | brain antigen 1 homolog) |
| | LBA1 | |
| B3KQL5 | | cDNA FLJ90678 fis, clone PLACE1005736, highly |
| | | similar to Pleckstrin homology domain-containing family |
| | | A member 1 |
| Q9HB21 | PLEKHA1 | Pleckstrin homology domain-containing family A |
| | TAPP1 | member 1 (PH domain-containing family A member 1) |
| | | (Tandem PH domain-containing protein 1) (TAPP-1) |
| O60264 | SMARCA5 | SWI/SNF-related matrix-associated actin-dependent |
| | SNF2H | regulator of chromatin subfamily A member 5 |
| | WCRF135 | (SWI/SNF-related matrix-associated actin-dependent |
| | | regulator of chromatin A5) (EC 3.6.4.—) (Sucrose |
| | | nonfermenting protein 2 homolog) (hSNF2H) |
| Q14789 | GOLGB1 | Golgin subfamily B member 1 (372 kDa Golgi complex- |
| | | associated protein) (GCP372) (Giantin) (Macrogolgin) |
| A0A087WUK2 | HNRNPDL | Heterogeneous nuclear ribonucleoprotein D-like |
| | HNRPDL | (Heterogeneous nuclear ribonucleoprotein D-like, |
| | hCG_22986 | isoform CRA_b) |
| O14979 | HNRNPDL | Heterogeneous nuclear ribonucleoprotein D-like (hnRNP |
| | HNRPDL | D-like) (hnRNP DL) (AU-rich element RNA-binding |
| | JKTBP | factor) (JKT41-binding protein) (Protein laAUF1) |

In one aspect, the active agent may be selected from one or more compounds as listed in Table 2.

TABLE 2
Compounds that inhibit proteins that inhibit nucleic acid
delivery vehicle uptake.

| Compound | | | CAS | Pubchem | |
|---|---|---|---|---|---|
| name | Structure | Source | registry | ID | PMID |
| Geldanamycin and derivative Alvespimycin | 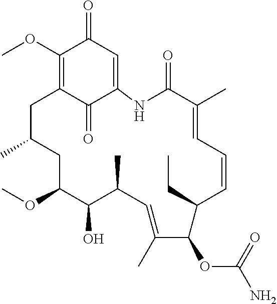 [SMILES: CC[C@H]1/C=C\C=C(/C)C(=O)NC2=CC(=O)C(OC)=C(C[C@@H](C)C[C@H](OC)[C@H](O)[C@@H](C)/C=C(\C)[C@@H]1OC(N)=O)C2=O] | Multiple, Wutech Acorn PharmaTech Procuct List ZINC OWNED by, Novartis | 30562-34- 6 | 5288382 | 1551101 2656616 |
| Entasobulin | 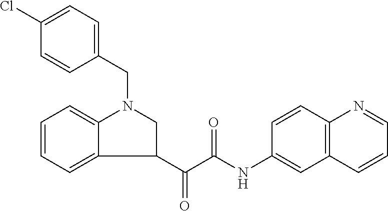 [SMILES: O=C(Nc1ccc2ncccc2c1)C(=O)C1CN(Cc2ccc(Cl)cc2)c2ccccc21] | Multiple ZINC MedChem express MCE ChemScene | 501921-61-5 | 10203597 | |
| Androstanolone/ Dihydrotestosterone | 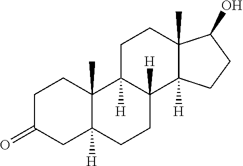 | Multiple, Sigma- Aldrich Key Organics/ BIONET 1717 CheMall Corporation OWNED by, | 12040-51- 6, 28801- 96-9, 29873-50- 5, 521-18- 6, 571-22- 2 | 10635, 15 | 106604538 20035615 20427476 |
| Spermine | 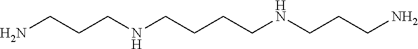 [SMILES: NCCCNCCCCNCCCN] | Multiple, Finetech Industry Limited | 77-44-3 | 1103 | 26962873 6534776 20427476 3878 |
| | | AK | | | |
| | | Scientific, | | | |
| | | Inc. | | | |
| | | (AKSCI) | | | |
| | | Sigma- | | | |
| | | Aldich | | | |
| Cortisone | 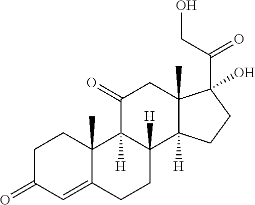 | Multiple, Ambinter LGC Standards AKos Consulting & Solutions | 53-06-5 | 222786 | 2427856655 |
| Qercetin | 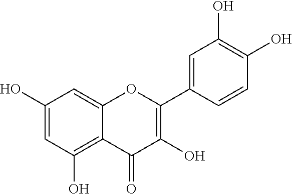 [SMILES: O=c1c(O)c(-c2ccc(O)c(O)c2)oc2cc(O)cc(O)c12] | Multiple, BePharm Ltd. Ambinter TimTec | 177-39-5, 6151-25- 3, 7255- 55-2, 73123-10- 1, 74893- 81-5 | 5280343 | 28574574 |
| Acetohexamide | 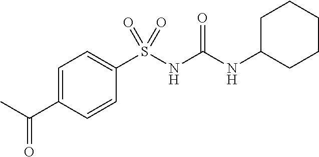 [SMILES: CC(=O)c1ccc(S(=O)(=O)NC(=O)NC2CCCCC2)cc1] | Multiple, TargetMol Boc Sciences Angene Chemical OWNED by, Watson Lilly | 8054-32- 8, 968-81- 0 | 1989 | 21249 22645689 |
| Resveratrol | 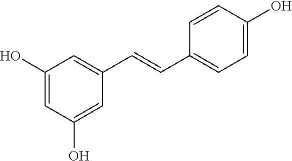 [SMILES: Oc1ccc(/C=C/c2cc(O)cc(O)c2)cc1] | Multiple, 1717 CheMall Corporation ApexBio Technology Sellckchem OWNED by, Home | 501-36-0 | 445154 | 7497631 28499732 28406974 |
| | | Aide | | | |
| | | Diagnostics, | | | |
| | | Inc. | | | |
| Doxorubicin | 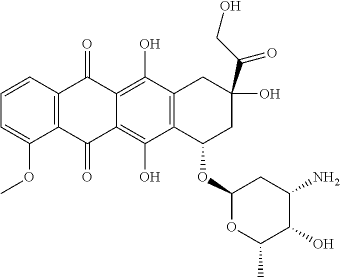 [SMILES: COc1cccc2c1C(=O)c1c(O)c3c(c(O)c1C2=O)C[C@@](O)(C(=O)CO)C[C@@H]3O[C@H]1C[C@H](N)[C@H](O)[C@H](C)O1] | Multiple, AbocChem LLC Alsachim ABBLIS Chemicals OWNED by, Pfizer | 23214-92- 8, 25316- 40-9 | 31703 | 3405 14644 28657372 28718370 |
| Ruxolitinib | 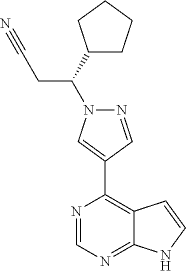 [SMILES: N#CC[C@H](C1CCCC1)n1cc(-c2ncnc3[nH]ccc23)cn1] | Multiple, BePharm Ltd. AvaChem Scientific Active Biopharma OWNED by, Novartis | 1092939- 17-7 | 25126798 | 19385672 19468275 28520871 |
| Roscovitine/ Seliciclib | 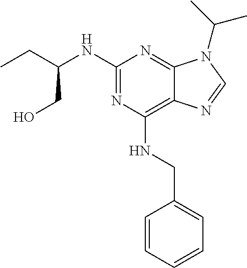 [SMILES: CC[C@H](CO)Nc1nc(NCc2ccccc2)c2ncn(C(C)C)c2n1] | Multiple, Tocris Bioscience abcr GmbH Boc Sciences OWNED by, Cyclacel Pharmaceuticals Inc. | 186692- 44-4 | 5097 | 26962873 9046330 20692737 |
| Sildenafil | 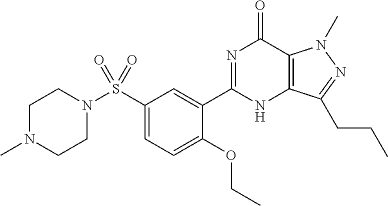 [SMILES: CCCc1nn(C)c2c(=O)nc(-c3cc(S(=O)(=O)N4CCN(C)CC4)ccc3OCC)[nH]c12] | Multiple, OXCHEM CORPOR- ATION MolPort Vitas-M Laboratory OWNED by, Pfizer Actavis Pharma Company | 139755- 83-2, 171599- 83-0 | 5212 | 28652262 28535536 28640077 |
| Teniposide/ Vumon | 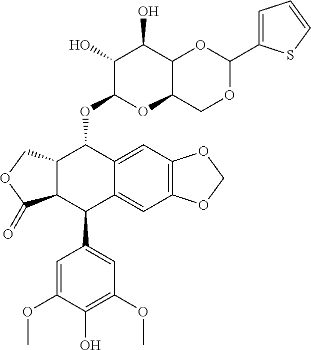 | Multiple, AK Scientific, Inc. AboveChem LLC BocSciences OWNED by, WG Critical Care, LLC Bristol Myers Squibb | 23362-13- 2, 29767- 20-2, 31514-29- 1, 35317- 44-3 | 34698 | 26916150 26583611 22771706 |

Description of agents in Table 2.

Geldanamycin is a benzoquinone ansamycin that binds to the heat shock protein Hsp90 and activates a heat shock response in mammalian cells.

Entasobulin is the first anticancer drug in development involving two mechanisms of action, tubulin and topoisomerase II inhibition. Entasobulin expresses different modes of action such as, pro-apoptotic and anti-angiogenic properties.

Dihydrotestosterone (DHT) (INN: androstanolone) is a biologically active metabolite of the hormone testosterone, formed primarily in the prostate gland, testes, hair follicles, and adrenal glands by the enzyme 5-alpha-reductase by means of reducing the alpha 4, 5 double-bond. Dihydrotestosterone belongs to the class of compounds called androgens, also commonly called androgenic hormones or testoids. DHT is thought to be approximately 30 times more potent than testosterone because of increased affinity to the androgen receptor.

Spermine is a polyamine involved in cellular metabolism found in all eukaryotic cells. The precursor for synthesis of spermine is the amino acid ornithine. It is found in a wide variety of organisms and tissues and is an essential growth factor in some bacteria. It is found as a polycation at physiological pH. Spermine is associated with nucleic acids and is thought to stabilize helical structure, particularly in viruses.

Cortisone is a Corticosteroid. The mechanism of action of cortisone is as a Corticosteroid Hormone Receptor Agonist.

Quercetin is a flavonoid and more specifically a flavonol and represents 60% of the total dietary flavonols intake. The term flavonoid comprises several thousand plant derived compounds sharing a common skeleton of phenyl-chromane. This basic structure allows a multitude of substitution patterns leading to several flavonoid subclasses such as flavonols, flavones, flavanones, catechins, anthocyanidins, isoflavones, dihydroflavonols and chalcones.

The first generation sulfonylureas include acetohexamide, chlorpropamide, tolazamide and tolbutamide, oral hypoglycemic agents that are used in therapy of type 2 diabetes.

Resveratrol (3,5,4′-trihydroxystilbene) is a polyphenolic phytoalexin. It is a stilbenoid, a derivate of stilbene, and is produced in plants with the help of the enzyme stilbene synthase. It exists as two structural isomers: cis-(Z) and trans-(E), with the trans-isomer shown in the top image. The trans-form can undergo isomerization to the cis-form when heated or exposed to ultraviolet irradiation. In a 2004 issue of Science, Dr. Sinclair of Harvard University said resveratrol is not an easy molecule to protect from oxidation. It has been claimed that it is readily degraded by exposure to light, heat, and oxygen. However, studies find that Trans-resveratrol undergoes negligible oxidation in normal atmosphere at room temperature.

Doxorubicin is a drug used in cancer chemotherapy. It is an anthracycline antibiotic, closely related to the natural product daunomycin, and like all anthracyclines it intercalates DNA. It is commonly used in the treatment of a wide range of cancers, including hematological malignancies, many types of carcinoma, and soft tissue sarcomas. The drug is administered in the form of hydrochloride salt intravenously. It may be sold under the brand names Adriamycin PFS, Adriamycin RDF, or Rubex. It is photosensitive and it is often covered by an aluminum bag to prevent light from affecting it.

Ruxolitinib (INCB018424) is a selective oral JAK1/JAK2 inhibitor. This agent has the potential to modulate two important kinases that may play a role in myeloproliferative neoplasms, including primary myelofibrosis.

Roscovitine is a Potent and Selective Inhibitor of the Cyclin-Dependent Kinases cdc2, cdk2 and cdk5.

Sildenafil is a selective PDE5 inhibitor that is used to treat erectile dysfunction and pulmonary arterial hypertension.

Teniposide/Vumon is a semisynthetic derivative of podophyllotoxin with antineoplastic activity. Teniposide forms a ternary complex with the enzyme topoisomerase II and DNA, resulting in dose-dependent single- and double-stranded breaks in DNA, DNA: protein cross-links, inhibition of DNA strand religation, and cytotoxicity. This agent acts in the late S or early G phase of the cell cycle.

TABLE 3
Compounds that inhibit proteins that inhibit nucleic acid delivery vehicle uptake

| # | Drug | Gene Symbol | Target | Effect | Pubmed |
|---|---|---|---|---|---|
| 1 | Entasobulin | TOP2B | TOP2 | Inhibition | |
| | intracellular | | beta | | |
| 2 | Memantine | GRIN3A | NR3A | Inhibition | 17157509 |
| | extracellular | | | | |
| | region | | | | |
| 3 | Teniposide | TOP2B | TOP2 | Inhibition | 8967966 |
| | intracellular | | beta | | |
| 4 | Etoposide | TOP2B | TOP2 | Inhibition | 1312600, 1312601, |
| | intracellular | | beta | | 1662724, 2158562, |
| | | | | | 2167985, 2537424, |
| | | | | | 2550587, 2849640, |
| | | | | | 7473578, 7922123, |
| | | | | | 8120864, 8295216, |
| | | | | | 8410993, 9211397, |
| | | | | | 10395485, 10809021, |
| | | | | | 11754608, 12877556, |
| | | | | | 15008514, 15084135, |
| | | | | | 15158802, 15177438, |
| | | | | | 16242334, 16903072, |
| | | | | | 17035025, 17580961, |
| | | | | | 14504921 |
| 5 | INO 1001 | PARP1 | PARP-1 | Inhibition | 15523000, 18535785, |
| | intracellular | | | | 20364863, 14523042 |
| 6 | Diazoxide | ABCC8 | SUR1 | Activation | 10419549, 11073882, |
| | intracellular | | | | 11121575, 12023875, |
| | | | | | 12565699, 14741296, |
| | | | | | 15561900 |
| 7 | Tedisamil | ABCC8 | SUR1 | Inhibition | 10445672, 10684468, |
| | extracellular | | | | 10829253 |
| | region | | | | |
| 8 | Glimepiride | ABCC8 | SUR1 | Inhibition | 9779817, 10773014, |
| | intracellular | | | | 11078468, 12819907, |
| | | | | | 20055691, 11325810 |
| 9 | Epirubicin | TOP2B | TOP2 | Inhibition | 16322310 |
| | intracellular | | beta | | |
| 10 | Annamycin | TOP2B | TOP2 | Inhibition | 15542779 |
| | intracellular | | beta | | |
| 11 | As(,2)O(,3) | PARP1 | PARP-1 | Inhibition | 12883267 |
| | intracellular | | | | |
| 12 | (R/S)- | ABCC8 | SUR1 | Inhibition | 10773014, 11440368, |
| | Repaglinide | | | | 11716850, 12196472, |
| | extracellular | | | | 12623163, 12819907, |
| | region | | | | 15200348, 15219283, |
| | | | | | 15380228, 15678092 |
| 13 | TOP53 | TOP2B | TOP2 | Inhibition | 11170388 |
| | intracellular | | beta | | |
| 14 | Acetohexamide | ABCC8 | SUR1 | Activation | 15200348, 15561903 |
| | extracellular | | | | |
| | region | | | | |
| 15 | Elsamitrucin | TOP2B | TOP2 | Inhibition | 8280493 |
| | intracellular | | beta | | |
| 16 | Ketamine | GRIN3A | NR3A | Inhibition | 17084865, 8336337, |
| | extracellular | | | | 8941398, 9719604, |
| | region | | | | 11937336 |
| 17 | NK109 | TOP2B | TOP2 | Inhibition | 9303354 |
| | intracellular | | beta | | |
| 18 | Tifenazoxide | ABCC8 | SUR1 | Activation | 12213059, 12961066, |
| | extracellular | | | | 14514634, 14764798, |
| | region | | | | 15220194 |
| 19 | Olaparib | PARP1 | PARP-1 | Inhibition | 18800822, 22343925, |
| | intracellular | | | | 23049934 |
| 20 | Intoplicine | TOP2B | TOP2 | Inhibition | 8043587 |
| | intracellular | | beta | | |

The gene transfer may occur in the context of administration to a cell in a human, i.e., administration of a vector containing a nucleic acid to a mammal, particularly a human. For example, an individual may be administered a compound and/or RNAi as disclosed herein prior to administration of a nucleic acid delivery system as known in the art (exemplary nucleic acid delivery systems are known in the art and disclosed in References 11-16). The nucleic acid may be single stranded or double stranded, or may, in certain instances, utilize multiple delivery vehicles which may employ one or the other or both.

The nanoparticle delivery vehicle may take a variety of forms. For example, in one aspect, the nucleic acid delivery vehicle may be a nanoparticle comprising said gene. In one aspect, the nucleic acid delivery vehicle may be a nanoparticle comprising a lysine polymer conjugated to PEG and complexed with a nucleic acid comprising the gene.

In one aspect, the proteins that inhibit the nucleic acid delivery vehicle uptake may be selected from keratin 13, APC protein, protocadherin 17, spectrin alpha (non-erythrocytic 1), or a combination thereof.

In one aspect, a period of time exists between step a and step b. In aspects in which the nucleic acid delivery vehicle is administered following delivery of an RNAi and/or compound as disclosed herein, the nucleic acid delivery vehicle may be administered to an individual in need thereof, for example, 30 minutes, or 60 minutes, or 90 minutes, or 120 minutes following the administration of a compound and/or RNAi as disclosed herein. In the case of RNAi, in some aspects, the RNAi may be administered about 12 hours in advance of a nucleic acid delivery vehicle, about 20 hours in advance of a nucleic acid delivery vehicle, about 24 hours in advance of a nucleic acid delivery vehicle, or about 30 hours in advance of administration of the delivery vehicle.

For example, for RNAi application, patient stem cells or patient derived iPSCs are harvested and cultured and treated with RNAi against a gene in Table 1 for 24 hr. NNPs formulated to contain an expression cassette for the therapeutic gene are then added to the cells for 72 hr. Reagents and delivery vector are replaced daily. An example of the time involved for the active agent application method is; patients are treated with one or more of the compounds claimed Tables 2 and 3 about 30 to about 60 minutes prior to gene delivery vector administration. Agent treatment may be conducted one or more times before gene therapy. NNPs containing an expression cassette for the therapeutic gene may then be administered to the airways of the patient, for example, via nebulization.

In one aspect, the method may include the step of providing a reagent that facilitates transfection. In one aspect, said agent may be a cationic lipid transfection reagent (e.g. Lipofectamine or GL67), which may be mixed with a nucleic acid under a given formulation to produce a nucleic acid/lipid complex. For lipid (or protein) nucleic acid complexes, any formulation that produces lipid/nucleic acid or protein/nucleic acid complexes (of which there are 1000s) can be combined with the methods herein. This may similarly apply to protein polymers such PEGylated poly L lysine or PEI. For viral vectors, the vector may be produced in cell lines, purified and used for therapy in accordance with the disclosed methods.

Compositions comprising RNAi and/or the compounds of Tables 2 and/or 3 may be administered intranasally. In such aspect, the compositions may further comprise other agents suited for improved delivery across nasal mucosa. For example, in certain aspects, agents such as a permeation enhancer, a polymer capable of increasing mucosal adhesion of the composition, or a combination thereof may be included in the composition.

It will be appreciated by those skilled in the art that the particular method of administration will depend on a variety of factors, all of which are considered routinely when administering therapeutics, particularly in the context of gene transfer. It will also be understood, however, that the specific dose level for any given patient will depend upon a variety of factors, including, the activity of the specific compound employed, the age of the patient, the body weight of the patient, the general health of the patient, the gender of the patient, the diet of the patient, time of administration, route of administration, rate of excretion, drug combinations, and the severity of the condition undergoing therapy. It will be further appreciated by one skilled in the art that the optimal course of treatment, i.e., the mode of treatment and the daily number of doses o given for a defined number of days, can be ascertained by those skilled in the art using conventional treatment tests.

### Kits

Kits are also provided. In one aspect, a kit may comprise or consist essentially of agents or compositions described herein. The kit may be a package that houses a container which may contain one or more compounds or solutions containing an RNAi as disclosed herein, and also houses instructions for administering the agent or composition to a subject. In one aspect, a pharmaceutical pack or kit is provided comprising one or more containers filled with one or more composition as disclosed herein. Associated with such container(s) can be various written materials such as instructions for use, or a notice in the form prescribed by a governmental agency regulating the manufacture, use or sale of pharmaceuticals or biological products, which notice reflects approval by the agency of manufacture, use, or sale for human administration.

As there may be advantages to mixing a component of a composition described herein and a pharmaceutically acceptable carrier, excipient or vehicle near the time of use, kits in which components of the compositions are packaged separately are disclosed. For example, the kit can contain an active ingredient in a powdered or other dry form in, for example, a sterile vial or ampule and, in a separate container within the kit, a carrier, excipient, or vehicle, or a component of a carrier, excipient, or vehicle (in liquid or dry form). In one aspect, the kit can contain a component in a dry form, typically as a powder, often in a lyophilized form in, for example, a sterile vial or ampule and, in a separate container within the kit, a carrier, excipient, or vehicle, or a component of a carrier, excipient, or vehicle. Alternatively, the kit may contain a component in the form of a concentrated solution that is diluted prior to administration. Any of the components described herein, any of the carriers, excipients or vehicles described herein, and any combination of components and carriers, excipients or vehicles can be included in a kit.

Optionally, a kit may also contain instructions for preparation or use (e.g., written instructions printed on the outer container or on a leaflet placed therein) and one or more devices to aid the preparation of the solution and/or its administration to a patient (e.g., one or a plurality of syringes, needles, filters, tape, tubing (e.g., tubing to facilitate intravenous administration) alcohol swabs and/or the Band-Aid® applicator). Compositions which are more concentrated than those administered to a subject can be prepared. Accordingly, such compositions can be included in the kits with, optionally, suitable materials (e.g., water, saline, or other physiologically acceptable solutions) for dilution. Instructions included with the kit can include, where appropriate, instructions for dilution.

In other embodiments, the kits can include pre-mixed compositions and instructions for solubilizing any precipitate that may have formed during shipping or storage. Kits containing solutions of one or more of the aforementioned active agents, or pharmaceutically acceptable salts thereof, and one or more carriers, excipients or vehicles may also contain any of the materials mentioned above (e.g., any device to aid in preparing the composition for administration or in the administration per se). The instructions in these kits may describe suitable indications (e.g., a description of patients amenable to treatment) and instructions for administering the solution to a patient.

### EXAMPLES

### Method for Enhancing Nucleic Acid Transfer

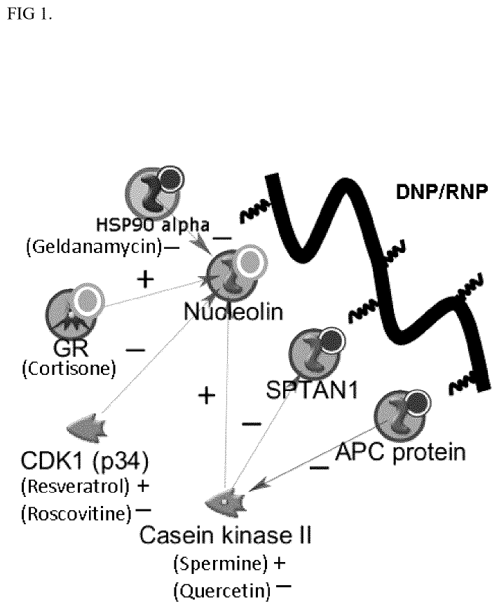

Applicant has discovered methods for enhancing the efficiency of gene transfer through the use of interference RNA (RNAi) technology or pharmacological agents that modulate the interactome (FIG. 1) of nucleic acid nanoparticles consisting of polymers of lysine conjugated to PEG and complexed with nucleic acids. Both of these approaches have been reduced to practice and achieve significantly higher levels of gene transfer in the context of condensed DNA nanoparticle vectors, resulting in as much as 50-fold greater gene transfer efficiency. These technologies represent a significant enhancement to gene transfer technologies.

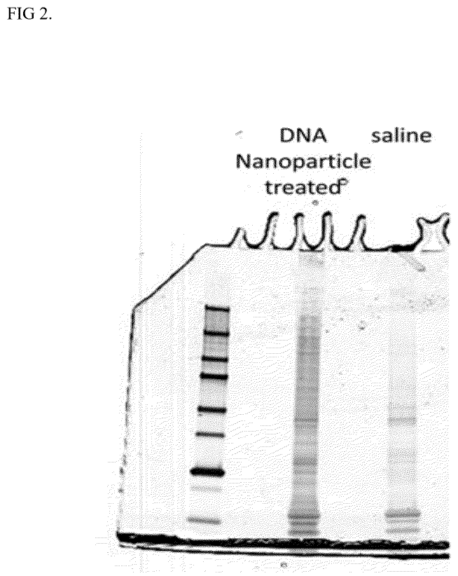

By using a novel immunocapture procedure (FIG. 2), Applicant identified protein interactors of polyethylene glycol conjugated DNA nanoparticles. This investigation revealed 474 unique proteins that interact with the nanoparticles as listed in Table 3. Many of these proteins represent a nanoparticle specific transfection interactome, but a number of proteins such as Prohibitin 1 and 2 are also involved in viral as well as liposomal gene delivery. Some of these protein interactors may be inhibiting the cellular uptake of DNA nanoparticles as well as other vectors for the delivery of nucleic acids. The interactome segregated into sites in the cell where nucleic acid particles are delivered (Table 4). In this method, Applicant used RNAi and/or pharmacological agents to modulate the particle interactome and enhance nucleic acid delivery to the nucleus (DNA) or the ribosome (RNA).

TABLE 4
Characteristics of the nucleic acid nanoparticle cellular protein interactome

| | | | | | | Percent of |
|---|---|---|---|---|---|---|
| | Cellular | | Cellular | | False Discovery | Dataset |
| Rank | Processes | Class | Localization | P Value | Rate | (%) |
| 1 | Intermediate | Cytoskeleton | Cytosol | 1.99E−14 | 8.68E−13 | 21.75 |
| | filaments | | | | | |
| 2 | Translation | Translation | Ribosome | 1.05E−16 | 1.37E−14 | 15.45 |
| | initiation | | | | | |
| 3 | Elongation- | Translation | Ribosome | 1.99E−15 | 1.30E−13 | 12.47 |
| | Termination | | | | | |
| 4 | Actin filaments | Cytoskeleton | Cytosol | 4.84E−08 | 1.58E−06 | 10.01 |
| 5 | Chromatin | Transcription | Nucleus | 7.80E−06 | 2.04E−04 | 9.71 |
| | modification | | | | | |
| 6 | Spindle | Cytoskeleton | Cytosol | 7.16E−04 | 8.53E−03 | 8.08 |
| | microtubules | | | | | |
| 7 | mRNA | Transcription | Nucleus | 1.06E−04 | 1.83E−03 | 7.71 |
| | processing | | | | | |
| 8 | Cell junctions | Cell adhesion | Cell | 1.21E−04 | 1.83E−03 | 7.61 |
| | | | membrane | | | |
| 9 | Regulation of | Cytoskeleton | Cytosol | 1.25E−04 | 1.83E−03 | 7.22 |
| | cytoskeleton | | | | | |
| | rearrangement | | | | | |

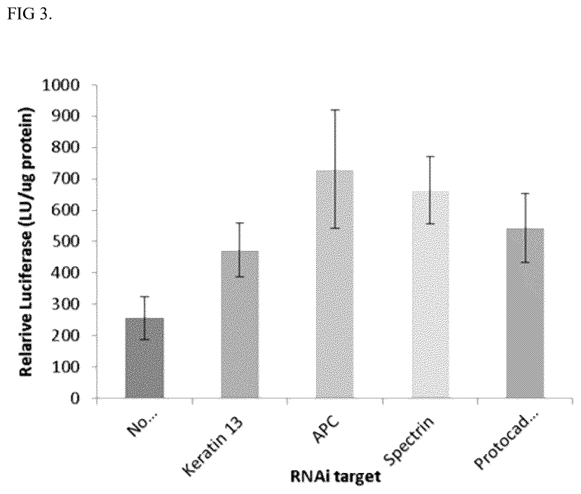

For RNAi application, RNAi molecules may be delivered to the cells, or in the case of delivery to an individual, to the individual, prior to the desired nucleic acid delivery vehicle. The RNAi molecules are administered in an amount sufficient to target and knock down specific cellular proteins that negatively impact the uptake of the nucleic acid delivery vehicle. RNAi decreases the cellular levels of these proteins, reducing their deleterious impact on the downstream transfer of nucleic acids. RNAi mediated knockdown of four of these proteins has been tested by Applicant, which resulted in significant enhancement of gene transfer in ¾ constructs tested. RNAi targeted to interfere with the synthesis of the 4 proteins; keratin 13 (GI: 81891678), APC protein (GI: 97535708), protocadherin 17 (GI:94538350), and spectrin alpha (non-erythrocytic 1, GI:119608216) that are deleterious to gene transfer with the DNA nanoparticles improved gene delivery (FIG. 3).

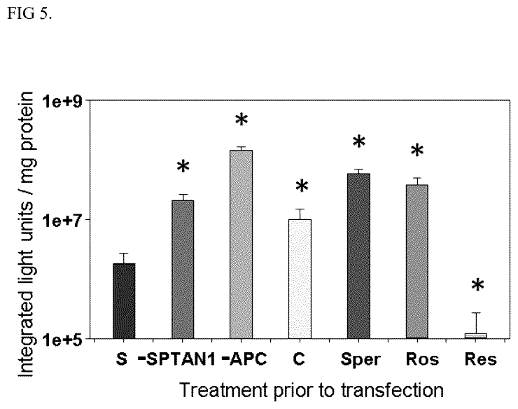

In addition or separately, pharmacological agents that modulate the DNP interactome can enhance nucleic acid transfer to the nucleus or the ribosome (in the case of RNA delivery). Applicant found 13 compounds and their derivatives that modulate 71 interactors (see Table 2) that can be administered to patients about 30 to about 60 minutes prior to dosing with DNPs. These are classified by cellular site of action. For example, Doxorubicin and Sildenafil will act to inhibit or promote interactions in the cytosol. Androstanolone will modulate interactions at the ribosome. Acetohexamide will promote non-nucleolin mediated interactions at the cell membrane. Ruxolitinib and Teniposide may be used to modulate nuclear interactions. Applicant's data also points to the importance to the interaction with nucleolin and how modulation of this interaction at the plasma membrane greatly impacts gene transfer with DNPs (FIGS. 3-5). Modulation of these cellular interactions is expected to have different impacts on RNPs vs. DNPs, as the cellular compartment targets for these formulations of NNPs vary (ribosome vs. nucleus, respectively). For example, drugs that promote cellular entry may benefit both DNPs and RNPs. However, drugs that diminish interactions at the ribosome would be expected to only benefit DNPs. Conversely, drugs that diminish nuclear interactions should benefit RNPs.

Table 2 and 3 outlines compounds may be used to modulate nucleolin associated nucleic acid nanoparticle (NNP) uptake. Nucleolin translocation to the cell surface may be promoted by IP injections of roscovitine (inhibits CDK1 at 10 mg/kg), spermine (induces CKII at 50 mg/kg), geldanamycin (inhibits HSP90 interaction with nucleolin at 15 mg/kg), or hydrocortisone (increases GR shuttling of nucleolin to the cell surface at 7.5 mg/kg) into animals 60 min prior to a 25 μl intranasal (IN) administration of 5 μg (with respect to DNA) NNPs containing the CFTR gene, as has been previously reported (1). Control groups injected with either DMSO instead of pharmacological agents, and NNPs containing the transgene with no drug may be used. CF mice may receive NPD measurements 1 week before treatment (a background/baseline measurement) and then 4, 7, and 14 days after transfection with CFTR-containing NNPs applied to the nose, as previously reported (1). Two weeks following transfection mice will be sacrificed, and the lungs may be harvested, paraffin imbeded, and sectioned for immunohistochemistry, and sections probed with the CF3 or 24:1 anti-CFTR Ab that does not cross react with mouse cftr, as previously reported (1). Studies can be duplicated in F508del and S489X homozygote mice.

### Use in Research

The RNAi and/or pharmacological approaches to enhancing gene transfer may be developed as an additive to current gene transfer and transfection vectors. For example, it may be used as a supplemental additive to the cationic lipid transfection reagent Lipofectamine, enabling either greater gene transfer or decreased amounts of transfection reagent used, resulting in either reduced costs or enhanced efficiency. Alternatively, pharmacological and RNAi treatment may be employed prior to or concurrent with the delivery of viral vectors in in vitro or ex vivo gene transfer applications. This may allow more cellular gene modification and higher expression of therapeutic transgenes within these cells, or decreased viral titer needed to provide curative levels of cell modification. This may increase the efficacy of these genes or reduce the associated costs with producing sufficient amount of virus, which is currently a significant obstacle in gene therapy protocols.

### Use in Human Therapy

CF is the most common inherited recessive disorders in Caucasians, and advances in small molecule therapy have not significantly benefitted a large majority of the patients. Gene therapy (repair or replacement) offers a potential of corrective therapy for the disease regardless of mutation type. The disclosed methods may be useful for enhancing corrective DNA and/or RNA delivery with a synthetic vector to airway epithelial cells, the most affected cells in CF. The present example relates to the biology of NNPs, a vector that has been shown to have partial efficacy in correcting CFTR in CF patients (2). Applicant has found 71 specific protein interactors (for example, some of the interactors and associated regulation are shown in FIG. 2, others are listed in Table 1) that help define the biology of the particles in cells and can be targeted with 13 FDA approved drugs (Table 2). Other compounds are listed in Table 3.

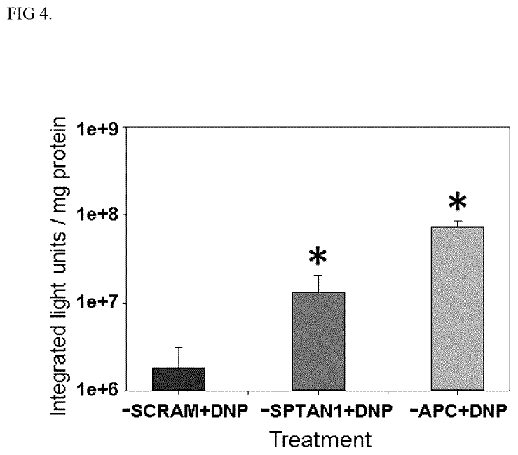

Applicant has demonstrated that modulating the NNP interactome can enhance gene transfer by 10-50 fold, the highest levels of enhancement ever achieved in two decades of modifying and examining DNP-based vectors (see FIGS. 3 and 4). Based on this result and given the fact that DNPs have achieved partial clinical correction in a Phase 1 trial in CF patients (2), the methods of the instant disclosure have the potential to provide pharmacological agents that can enhance gene transfer to fully therapeutic levels in humans. While airway epithelial cells are the primary site of disease and the most important gene therapy target in CF, a better understanding of the determinants of successful gene transfer into these cells will significantly benefit gene delivery for a number of other diseases, including chronic obstructive pulmonary disease (COPD; ˜12,000,000 patients in the USA), and epithelial lung cancers (˜200,000 patients in the USA). The instant disclosure provides a novel approach to implementation of NNP biology. Findings in airway epithelia will likely be relevant to other cell targets where NNPs have succeeded, including cells in the brain (3-6) and retina (7-10), and may be relevant to the cellular uptake of other non-viral polyplex-based vectors as well as viral and liposomal vectors.

In a therapeutic context, siRNA can be applied to human cells ex vivo or pharmacological agents to humans directly before or during gene delivery to optimize gene transfer obtained with DNA/RNA nanoparticles, and potential with liposomal and viral vectors as well.

### Example of Application in Cystic Fibrosis

For RNAi application, patient stem cells or patient derived iPSCs are harvested and cultured and treated with RNAi against keratin 13 (GI: 81891678), APC protein (GI: 97535708), protocadherin 17 (GI:94538350), and spectrin alpha (non-erythrocytic 1, GI:119608216) for 24 hr. NNPs formulated to contain an expression cassette for the cystic fibrosis transmembrane conductance regulator (CFTR), the protein mutated in cystic fibrosis, are added to cells for 72 hr. A fraction of cells is tested for CFTR expression, any integration events, and health and morphology, and the remainder of the cells is transferred to the patient.

For pharmacological application, patients may be treated with one or more of the compounds claimed about 30 to about 60 minutes prior to NNP administration. Agent treatment may be conducted once before gene therapy. NNPs contacting an expression cassette for the CFTR gene may be administered to the airways of the patient by nebulization, and gene transfer may be monitored in follow up examinations. The claimed agents may also enhance the delivery of other gene transfer vectors such as liposomal and viral vectors. Pharmacological application may be carried out in combination with RNAi treatment of cells harvested or derived from patients.

### REFERENCE LIST

1. Ziady AG, Kelley TJ, Milliken E, Ferkol T, Davis PB. Functional evidence of CFTR gene transfer in nasal epithelium of cystic fibrosis mice in vivo following luminal application of DNA complexes targeted to the serpin-enzyme complex receptor. Mol.Ther. 2002 Apr;5(4):413-9
2. Konstan MW, Davis PB, Wagener JS, Hilliard KA, Stern RC, Milgram LJ, Kowalczyk TH, Hyatt SL, Fink TL, Gedeon CR, et al. Compacted DNA nanoparticles administered to the nasal mucosa of cystic fibrosis subjects are safe and demonstrate partial to complete cystic fibrosis transmembrane regulator reconstitution. Hum.Gene Ther. 2004 Dec;15(12):1255-69
3. Yurek DM, Fletcher AM, Smith GM, Seroogy KB, Ziady AG, Molter J, Kowalczyk TH, Padegimas L, Cooper MJ. Long-term transgene expression in the central nervous system using DNA nanoparticles. Mol.Ther. 2009 Apr;17(4):641-50
4. Yurek DM, Flectcher AM, Kowalczyk TH, Padegimas L, Cooper MJ. Compacted DNA nanoparticle gene transfer of GDNF to the rat striatum enhances the survival of grafted fetal dopamine neurons. Cell Transplant. 2009;18(10):1183-96. PMCID:PMC3031110
5. Yurek DM, Fletcher AM, McShane M, Kowalczyk TH, Padegimas L, Weatherspoon MR, Kaytor MD, Cooper MJ, Ziady AG. DNA Nanoparticles: Detection Of Long-term Transgene Activity In Brain Using Bioluminescence Imaging. Mol.Imaging 2011 Apr 26;
6. Fletcher AM, Kowalczyk TH, Padegimas L, Cooper MJ, Yurek DM. Transgene expression in the striatum following intracerebral injections of DNA nanoparticles encoding for human glial cell line-derived neurotrophic factor. Neuroscience 2011 Oct 27;194:220-6. PMCID:PMC3408714
7. Farjo R, Skaggs J, Quiambao AB, Cooper MJ, Naash MI. Efficient non-viral ocular gene transfer with compacted DNA nanoparticles. PLOS.One. 2006;1:e38. PMCID:PMC1762345
8. Ding XQ, Quiambao AB, Fitzgerald JB, Cooper MJ, Conley SM, Naash MI. Ocular delivery of compacted DNA-nanoparticles does not elicit toxicity in the mouse retina. PLOS.One. 2009;4(10):e7410. PMCID:PMC2756629
9. Cai X, Conley SM, Nash Z, Fliesler SJ, Cooper MJ, Naash MI. Gene delivery to mitotic and postmitotic photoreceptors via compacted DNA nanoparticles results in improved phenotype in a mouse model of retinitis pigmentosa. FASEB J. 2010 Apr;24(4):1178-91. PMCID:PMC2845431
10. Koirala A, Makkia RS, Conley SM, Cooper MJ, Naash MI. S/MAR-containing DNA nanoparticles promote persistent RPE gene expression and improvement in RPE65-associated LCA. Hum.Mol.Genet. 2013 Apr 15;22(8):1632-42. PMCID:PMC3605833
11. Ziady AG, Davis PB, Konstan MW. Non-viral gene transfer therapy for cystic fibrosis. Expert.Opin.Biol.Ther. 2003 Jun;3(3):449-58
12. Ziady AG, Davis PB. Current prospects for gene therapy of cystic fibrosis. Curr.Opin.Pharmacol. 2006 Oct;6(5):515-21
13. Ahmed H, Shubina-Oleinik O, Holt JR. Emerging Gene Therapies for Genetic Hearing Loss. J.Assoc.Res.Otolaryngol. 2017 Aug 16;
14. Naso MF, Tomkowicz B, Perry WL, III, Strohl WR. Adeno-Associated Virus (AAV) as a Vector for Gene Therapy. BioDrugs. 2017 Jul 1. PMCID:PMC5548848
15. Huang J, Wang Y, Zhao J. CRISPR Editing in Biological and Biomedical Investigation. J.Cell Physiol 2017 Aug 8;
16. Zhang X, Wang L, Liu M, Li D. CRISPR/Cas9 system: a powerful technology for in vivo and ex vivo gene therapy. Sci.China Life Sci. 2017 May;60(5):468-75

All percentages and ratios are calculated by weight unless otherwise indicated.

All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1-17. (canceled)

18. An article of manufacture comprising a sterile container containing a composition wherein the composition comprises
a. an RNAi molecule that inhibits expression of a gene encoding a protein, the protein being selected from Apolipoprotein B-100 (Apo B-100), Leiomodin-1, Elongation factor 1-alpha 1, Pot. HLA-DP-alpha 1, Utrophin, Myosin light chain 3, Cytochrome b-c1 complex subunit 2, Ubiquitin-conjugating enzyme E2 S, Coagulation factor VIII, Heterogeneous nuclear ribonucleoprotein M, Myosin light polypeptide 6, Adenomatous polyposis coli protein, Tyrosine-protein kinase JAK1, Myosin-6, 40S ribosomal protein S3a, Golgin subfamily A member 2, Peripherin (Neurofilament 4), Heterogeneous nuclear ribonucleoprotein A/B, Spectrin beta chain, erythrocytic, Dual specificity protein kinase TTK, 78 kDa glucose-regulated protein, CACCC box-binding protein, 60S ribosomal protein L11, Eukaryotic initiation factor 4A-III, Interleukin enhancer-binding factor 2, Nucleolar and coiled-body phosphoprotein 1, Nebulin, Microtubule-associated protein, DNA replication licensing factor MCM4, Centromere protein F (CENP-F), Cytoskeleton-associated protein 5, Chromodomain-helicase-DNA-binding protein 4, Solute carrier family 12 member 3, Phosphatidylinositol 3,4,5-trisphosphate 5-phosphatase 1, Periodic tryptophan protein 2 homolog, DNA mismatch repair protein Msh3, Ubiquitin-protein ligase E3A, SWI/SNF complex subunit SMARCC1, Histone H2B type 1-C/E/F/G/I, Histone-lysine N-methyltransferase EZH1, Endonuclease III-like protein 1, General transcription factor 3C polypeptide 1, Histone-lysine N-methyltransferase 2D (Lysine N-methyltransferase 2D), Uracil nucleotide/cysteinyl leukotriene receptor, Catenin delta-2, Heme oxygenase 2, Periplakin, Ryanodine receptor 3, Cullin-3, Small subunit processome component 20 homolog, T13D8.9 protein, RNA-binding motif protein, X chromosome, Protein CBFA2T3, Peripheral-type benzodiazepine receptor-associated protein 1, Actin, gamma-enteric smooth muscle, Regulator of chromosome condensation, CHC1 protein, Elongation factor 2, Histone H3.1t, Prohibitin, isoform CRA_a, Prohibitin, Prohibitin variant, 60S ribosomal protein L24, Epididymis secretory protein Li 310, Ribosomal protein, large, P2, 60S acidic ribosomal protein P2, 40S ribosomal protein S10, 40S ribosomal protein S11, 40S ribosomal protein S13, 40S ribosomal protein S17, Ribosomal protein S23, isoform CRA_a, 40S ribosomal protein S23, 40S ribosomal protein S25, 40S ribosomal protein S4, 40S ribosomal protein S4, X isoform, 40S ribosomal protein S8, Ski oncogene (Proto-oncogene c-Ski), Sperm binding protein 1a, a protein encoded by cDNA FLJ38670 fis, clone HSYRA2000190, highly similar to Voltage-dependent anion-selective channel protein 1, Voltage-dependent anion-selective channel protein 1 (VDAC-1), Transcriptional repressor protein YY1, A-kinase anchor protein 9, Histone H1.3, Zinc finger C2HC domain-containing protein 1A, Prelamin-A/C, Lamin-B1, Alcohol dehydrogenase, Epididymis secretory protein Li 6, ATP5H protein, ATP synthase subunit d, mitochondrial, Myosin regulatory light chain 12A, Uncharacterized protein, Gag-Pro-Pol-Env protein, High mobility group protein HMG-I/HMG-Y, Cytochrome c oxidase subunit 4 isoform 1, mitochondrial, Tyrosine-protein kinase BAZ1B, Septin-9, 40S ribosomal protein S18, MCG23000, isoform CRA_b, Required for meiotic nuclear division protein 1 homolog, Heterogeneous nuclear ribonucleoprotein H3, Low-density lipoprotein receptor-related protein 1B, MICOS complex subunit MIC60, MICOS complex subunit, MICOS complex subunit MIC19, Rhomboid domain-containing protein 2, Armadillo repeat-containing X-linked protein 5, ATP-dependent RNA helicase DDX39A, Leucine-rich repeat-containing protein 31, Coiled-coil domain-containing protein 170, ABC50 protein, ATP-binding cassette sub-family F member 1, Cell division cycle 5-like protein, Myosin-10, 60S ribosomal protein L14, Sperm flagellar protein 2, Keratin, type II cytoskeletal 7, Similar to ribosomal protein L23, Gamma-tubulin complex component 6, Ribosomal protein L15, 60S ribosomal protein L15, KLHL20 protein, High mobility group nucleosome-binding domain-containing protein 5, Nucleolar protein 7, 60S ribosomal protein L10a, DNA-dependent protein kinase catalytic subunit, a protein encoded by cDNA FLJ51732, highly similar to Peroxisomal NADH pyrophosphatase NUDT12 (EC 3.6.1.22), Peroxisomal NADH pyrophosphatase NUDT12, 60S ribosomal protein L28, Heterogeneous nuclear ribonucleoproteins A2/B1, Serine/threonine-protein kinase SMG1, 40S ribosomal protein S9, Remodeling and spacing factor 1 (Rsf-1), Actin, cytoplasmic 1, BRCA1-A complex subunit RAP80, Galactose-3-O-sulfotransferase 3, 40S ribosomal protein S24, Bromodomain and WD repeat-containing protein 1, Junction plakoglobin, isoform CRA_a, Membrane-associated guanylate kinase, WW and PDZ domain-containing protein 1, 60S ribosomal protein L13, Phosphorylase b kinase regulatory subunit alpha, Heterogeneous nuclear ribonucleoprotein Q, Alsin, Synaptotagmin-like protein 1, Ribosomal protein L34, 60S ribosomal protein L34, Cingulin, Centromere protein T, Cingulin-like protein 1, Coiled-coil domain-containing protein 42, Heterogeneous nuclear ribonucleoprotein F, Mitochondrial import receptor subunit TOM40 homolog, FBL protein, ESF1 homolog, Calcium/calmodulin-dependent protein kinase type II subunit delta, 60S ribosomal protein L18, Protein Shroom3, Dynein heavy chain 5, axonemal, PC4 and SFRS1-interacting protein, Epididymis tissue sperm binding protein Li 14m, Trifunctional enzyme subunit alpha, mitochondrial, Bcl-2-like protein 12, Core histone macro-H2A.1, MTRF1 protein, Alstrom syndrome protein 1, UHRF1-binding protein 1-like, U5 small nuclear ribonucleoprotein 200 kDa helicase, SWI/SNF related, matrix associated, actin dependent regulator of chromatin, subfamily a, member 4, Transcription activator BRG1, Eukaryotic elongation factor 2 kinase, EVI5-like protein, Protein ECT2, CSC1-like protein 2, a protein encoded by cDNA FLJ35251 fis, clone PROST2003635, Nesprin-1, Nesprin-1, Chromodomain-helicase-DNA-binding protein 5, Midasin, Nesprin-2, Voltage-dependent anion-selective channel protein 3, ATPase family AAA domain-containing protein 5, N-alpha-acetyltransferase 15, NatA auxiliary subunit, Histone H2A type 2-B, Rootletin, Histone H2A, Histone H2A.J, PDZ domain-containing protein 8, Structural maintenance of chromosomes protein 1A, SMC1A protein, Vacuolar protein sorting-associated protein 13B, Centriolin, Pericentrin, Testicular secretory protein Li 14, Glutamate dehydrogenase 2, Myomesin-3, Acidic ribosomal phosphoprotein P1, Nucleolar protein 56, Nucleolar protein 58, Uncharacterized protein, FLJ00279 protein, a protein encoded by cDNA FLJ16045 fis, clone CTONG2000042, Probable C-mannosyltransferase DPY19L4, Coiled-coil domain-containing protein 18, FYVE, RhoGEF and PH domain-containing protein 6, ATP synthase subunit alpha, mitochondrial, Superkiller viralicidic activity 2-like 2, Phosphate carrier protein, mitochondrial, 40S ribosomal protein S6, EEF1D protein (Fragment), Sterile alpha motif domain-containing protein 9, Antigen MLAA-44, Coiled-coil and C2 domain-containing protein 1B, Elongation factor 1-gamma, Heterogeneous nuclear ribonucleoprotein U, Malignant fibrous histiocytoma-amplified sequence 1, Arachidonate 15-lipoxygenase, GC-rich sequence DNA-binding factor 2, 60S ribosomal protein L4, Regulator of G-protein signaling 20, Harmonin, Plectin, Kinesin-like protein KIF1B (Klp), Neuropilin-2, Neuropilin, Vacuolar protein sorting-associated protein 13D, Lymphoid-specific helicase, Histone H2B type 1-A, Ankyrin repeat domain-containing protein 11, Histone H2B (Fragment), Keratin, type II cytoskeletal 6A, Nipped-B-like protein (Delangin) (SCC2 homolog), ATPase family AAA domain-containing protein 1, Mitochondrial dynamin-like 120 kDa protein, Striated muscle preferentially expressed protein kinase, Disks large-associated protein 1, Dedicator of cytokinesis protein 11, Myomegalin, Putative uncharacterized protein DKFZp666F1010, Putative uncharacterized protein DKFZp666M0710, Putative uncharacterized protein DKFZp686P17171, Activation-dependent, raft-recruited ADAP-like phosphoprotein, Unconventional myosin-XVIIIa, Nucleolin, isoform CRA_b, Nucleolin, isoform CRA_c, Nucleolin (Protein C23), Keratin, type I cytoskeletal 9, Cilia- and flagella-associated protein 58, Terminal uridylyltransferase 4, WD repeat and SOCS box-containing protein 1, Centromere protein J, FRAS1-related extracellular matrix protein 1, Macoilin, NARG1 protein (Fragment), Calpastatin isoform a variant (Fragment), Ribosomal protein L5 variant (Fragment), Insulin-like growth factor binding protein 2 variant (Fragment), Rho-associated protein kinase 2, 60S acidic ribosomal protein P0 (Fragment), Elongation factor 1-alpha (Fragment), Glial fibrillary acidic protein, Beta-actin-like protein 2, Fanconi anemia, complementation group D2, isoform CRA_b, Fanconi anemia group D2 protein, IQ and AAA domain-containing protein 1, Protein FAM92A, Teneurin-3, Coiled-coil and C2 domain-containing protein 2A, Putative uncharacterized protein C3orf49, Heterogeneous nuclear ribonucleoprotein A1, Dynein heavy chain 2, axonemal, Ribosomal protein (Fragment), RSL1D1 protein (Fragment), ATP-dependent DNA helicase PIF1, Keratin, type II cytoskeletal 73, ATP synthase subunit beta, a protein encoded by cDNA FLJ39263 fis, clone OCBBF2009571, ATP-dependent RNA helicase A, Centrosomal protein of 290 kDa, CEP290 protein, Golgi-specific brefeldin A-resistance guanine nucleotide exchange factor 1, La-related protein 7, Pumilio homolog 3, Ovochymase-1, INO80 complex subunit, RWD domain-containing protein 3, Dual 3′,5′-cyclic-AMP and -GMP phosphodiesterase 11A, Histone-lysine N-methyltransferase ASH1L, ATP-binding cassette sub-family C member 8, Cilia- and flagella-associated protein 157, M-phase phosphoprotein 1, isoform CRA_a, 60S ribosomal protein L3, A kinase (PRKA) anchor protein 6, isoform CRA_b, Lamin B2, isoform CRA_b, Nuclear mitotic apparatus protein 1, isoform CRA_a, Coiled-coil domain-containing protein 40, TBK1 binding protein 1, isoform CRA_a, Lamina-associated polypeptide 2, isoforms beta/gamma, KIAA1212, isoform CRA_a, Histone H2B, Septin 11, isoform CRA_b, Heterogeneous nuclear ribonucleoprotein A3, isoform CRA_a, MORC family CW-type zinc finger protein 4, Cytoplasmic dynein 2 heavy chain 1, Unconventional myosin-VIIb, Protein unc-13 homolog C, HSPA1L, Heat shock 70 kDa protein 1-like, Laminin subunit beta-4, Leucine-rich repeat-containing protein 43, Protocadherin Fat 3, Death-associated protein kinase, ADP/ATP translocase 2, SLC25A5 protein (Fragment), ADP/ATP translocase 3, SLC25A6 protein, HNRPR protein (Fragment), Poly [ADP-ribose] polymerase, Poly [ADP-ribose] polymerase 1, Protein AHNAK2, Myb-binding protein 1A, Leucine-rich repeat and IQ domain-containing protein 3, a protein encoded by cDNA FLJ76880, a protein encoded by CDNA FLJ76071, highly similar to Homo sapiens filamin A interacting protein 1 (FILIP1), mRNA, Structural maintenance of chromosomes protein, Coiled-coil domain-containing protein 180, Dynein heavy chain 17, axonemal, Histone H2A, a protein encoded by cDNA, FLJ95007, highly similar to Homo sapiens BRCA1 associated RING domain 1 (BARD1), mRNA, Protein fantom, Mpv17-like protein, Protein CASP, a protein encoded by cDNA FLJ44920 fis, clone BRAMY3011501, highly similar to Heterogeneous nuclear ribonucleoprotein U, ATPase family AAA domain-containing protein 3A, a protein encoded by cDNA FLJ35376 fis, clone SKMUS2004044, highly similar to Homo sapiens ribosomal protein L3 (RPL3), transcript variant 2, mRNA, SNF2LT, Dynein heavy chain 6, axonemal, Nuclear export mediator factor NEMF, a protein encoded by cDNA FLJ51361, highly similar to Keratin, type II cytoskeletal 6A, a protein encoded by cDNA FLJ57651, highly similar to Ketosamine-3-kinase, a protein encoded by cDNA FLJ50007, a protein encoded by cDNA FLJ52940, highly similar to Mortality factor 4-like protein 2, Protein RUFY3, Leucine-rich repeat and coiled-coil domain-containing protein 1, a protein encoded by cDNA FLJ60374, G-protein coupled receptor 176, a protein encoded by cDNA FLJ61500, highly similar to NNP-1 protein, Small nuclear ribonucleoprotein Sm D3, a protein encoded by cDNA FLJ54081, highly similar to Keratin, type II cytoskeletal 5, a protein encoded by cDNA FLJ58017, moderately similar to Treacle protein, Piwi-like protein 2, Serine/threonine-protein kinase N2, Dysferlin, Putative uncharacterized protein DKFZp469P0721, Short transient receptor potential channel 4, GTP-binding nuclear protein Ran, HNRNPL protein (Fragment), a protein encoded by cDNA FLJ52713, moderately similar to Mus musculus leucine rich repeat (in FLII) interacting protein 1 (Lrrfip1), mRNA, a protein encoded by cDNA FLJ50492, highly similar to Cyclin-dependent kinase-like 3 (EC 2.7.11.22), TBC1 domain family member 1, Non-POU domain-containing octamer-binding, 60S ribosomal protein L31, a protein encoded by CDNA FLJ51382, Protein Jumonji, von Willebrand factor A domain-containing protein 5B2, VWA5B2 protein (Fragment), Centrosomal protein of 128 kDa (Cep128), Proliferation marker protein Ki-67, Ribosomal protein L19, RPL27/NME2 fusion protein (Fragment), TPR and ankyrin repeat-containing protein 1, a protein encoded by cDNA FLJ90678 fis, clone PLACE1005736, highly similar to Pleckstrin homology domain-containing family A member 1, Pleckstrin homology domain-containing family A member 1, SWI/SNF-related matrix-associated actin-dependent regulator of chromatin subfamily A member 5, Golgin subfamily B member 1, Heterogeneous nuclear ribonucleoprotein D-like, and combinations thereof; and
b. an active agent of selected from geldanamycin, alvespimycin, androstanolone, spermine, cortisone, quercetin, acetohexamide, resveratrol, doxorubicin, ruxolitinib, roscovitine, sildenafil, entasobulin, memantine, teniposide, etoposide, 3-aminobenzamide (INO 1001), diazoxide, tedisamil, glimepiride, epirubicin, annamycin, As₂O₃, (R/S)-repaglinide, 4β-aminoalkyl-4′-O-demethyl-4-desoxypodophyllotoxin (TOP53), acetohexamide, elasmitrucin, ketamine, NK109, tifenazoxide, olaparib, intoplicine, and combinations thereof.

19. The article of claim 18, wherein the RNAi molecule and the active agent are separate.

20. The article of claim 18, wherein one or both of the active agent and the RNAi are provided in a dry form.

21. The article of claim 18, wherein one or both of the active agent and the RNAi are provided in a lyophilized powder.

22. The article of claim 18, wherein one or both of the active agent and the RNAi are provided in a liquid form.

23. The article of claim 18, wherein one or both of the active agent and the RNAi are in the form of a concentrated solution.

24. The article of claim 23, further comprising a diluent diluting the concentration solution.

25. The article of claim 42, further comprising instructions for diluting the concentrated solution prior to administration.

26. The article of claim 18, further comprising a device for preparing the composition.

27. The article of claim 26, wherein the device is selected from one or more of a syringe, a needle, a filter, a tubing, and an alcohol swab.

28. The article of claim 18, further comprising a device for administering the composition.

29. The article of claim 28, wherein the device is selected from one or more of a syringe, a needle, a filter, a tubing, and an alcohol swab.

30. The article of claim 18, further comprising instructions for preparation or use.

31. The article of claim 30, wherein the instructions are printed on an outer container.

32. The article of claim 18, further comprising saline.

33. The article of claim 18, wherein the sterile container is a vial or ampule.

34. The article of claim 18, wherein the RNAi molecule and/or active agent is in a pre-mixed composition comprising one or more of a carrier, an excipient, or a vehicle.

35. The article of claim 18, further comprising written instruction comprising a description of patients amenable to treatment.
